(19) United States
(12) Patent Application Publication
(10) Pub. No.: US 2024/0398717 A1
(43) Pub. Date: Dec. 5, 2024

(54) Method of using binary coating agents in powder coating to achieve enhanced properties of the coated powder and its blends having cohesive powders

(71) Applicant: New Jersey Institute of Technology, Newark, NJ (US)

(72) Inventors: Rajesh N. Dave, Newark, NJ (US); Sangah Selene Kim, Newark, NJ (US); Zhixing Lin, Newark, NJ (US)

(73) Assignee: New Jersey Institute of Technology, Newark, NJ (US)

(21) Appl. No.: 18/789,155

(22) Filed: Jul. 30, 2024

Related U.S. Application Data
(63) Continuation-in-part of application No. 18/221,143, filed on Jul. 12, 2023, now Pat. No. 12,076,440.
(60) Provisional application No. 63/388,422, filed on Jul. 12, 2022.

(51) Int. Cl. A61K 9/50

(52) U.S. Cl. CPC A61K 9/501; A61K 9/5042; A61K 9/5089

(57) ABSTRACT

A method of employing a mixture of two different types of silica as a binary dry coating material to benefit properties of fine milled pharmaceuticals, such as ibuprofen, milled to the mean particle size of ˜10 μm or smaller. Synergistic effects of reduction in agglomerate size, and improved surface wettability was found by employing a mixture of two different types of silica as the dry coating material in specific ratios. The silica used up to weight percent to 2.31 wt % of a minority component, typically an API or excipient. The silica amount in the entire blend was less than 0.1 wt %. The surface coverage of API was optimized to 50%. Properties were increased when surface coverage by binary coating agents was controlled so that no excess coating agent was available to form agglomerates on the surface.

### CROSS-REFERENCE TO RELATED APPLICATIONS

The present application is a continuation-in-part of U.S. Application No. 18/221,143 filed Jul. 12, 2023, now allowed, that claims the benefit of the filing date of U.S. Provisional Application No. 63/388,422 filed Jul. 12, 2022, the disclosures of which are hereby incorporated herein by reference.

### GOVERNMENT STATEMENT

This invention was made with government support under contract grant number IIP-1919037, titled “PFI-RP: Commercializing innovations in design and manufacturing of fine pharmaceutical powders for cheaper and better medicines,” and awarded by the National Science Foundation (NSF). The government has certain rights in the invention.

### FIELD OF THE DISCLOSURE

The present disclosure relates to use of binary coating agents with differing properties such as hydrophilic and hydrophobic silicas simultaneously to coat a substrate or host particles to achieve unexpectedly enhanced properties for the powder and its blends. Some of the noteworthy property improvements for otherwise cohesive powders include enhanced flowability, reduced agglomeration, increased bulk and density faster dissolution as compared to not using the combination of coating agents. The present disclosure also relates to any coated component of blends, including API and/or excipients, and more particularly to dry surface coating of binary or multi-component blends having low coated component loading of less than 1% wt with the coating agents.

### BACKGROUND

Formulators in pharmaceutical, nutraceutical and other industries face significant challenges that deal with preparing multi-constituent blends of active ingredient mixed with other functional materials. For example, in pharmaceutical industry, significant amount of development time, materials, and effort is required for developing tablet or capsule formulations that exhibit desirable properties such as flowability, bulk packing density, compressibility or compactability, blend content uniformity, as well as dissolution from the API powders, without having to resort to granulation, especially wet granulation that consists of multiple steps and use of liquids.

This problem is even more challenging and almost insolvable without resorting to dry or wet granulation when the active ingredients are fine sized powders, ranging from 10 to 70 micron, usually further categorized as fine (10-15 micron) or semi-fine (30-50 micron). While it is generally known that when the blend includes flow aids such as fumed silica, the blend properties such as flowability may be improved provided that the flow aid is of a significant amount for example much greater than 1 wt % of the blend. However, increased use of silica decreases some important properties required for forming a tablet, capsule, or sachet. In addition it remains a challenge to achieve simultaneous improvements in all such properties while using lesser amounts of silica and when the APIs (or active ingredients in other industry applications) are fine.

Blend flowability and API release rate from the final product form (tablet, capsule, or sachet) are affected by the hydrophobicity of the selected coating agents. Hence, a hydrophilic coating agent is often preferred choice over a hydrophobic coating agent despite lesser bulk properties (flowability, bulk density, deagglomeration) enhancement than hydrophobic coating agent to avoid potential delays in APIs' dissolution or release rate.

Again, drug products such as compressed tablets, filled capsules or sachets, require blends, which include drug and excipients, which have desired quality attributes, such as adequate flowability, bulk density, and drug content uniformity, to name a few. Amongst those, flowability is a critical factor that is an indicator of successful manufacturing and product quality of tablets (Sun CC. Setting the bar for powder flow properties in successful high speed tableting. Powder Technology. 2010; 201 (1): 106-108), capsules (Tan, S. and J. Newton, Powder flowability as an indication of capsule filling performance. International Journal of Pharmaceutics, 1990. 61 (1-2): p. 145-155), and powder filled sachet (Pitt K. Formulation and processing for powder sachets. In: GD, Tovey editor. Specialized pharmaceutical formulation: Royal Society of Chemistry; 2022. p. 338).

As micronized, finer sized, e.g., 30 mm and lower, drug powders are increasingly being used for assorted reasons including improved drug dissolution, achieving desired blend properties gets more challenging. Even the use of well flowing or specialized excipients, e.g., silicified MCC has been found to be insufficient in imparting such desired qualities (L. Chen, S. Fan, Z. He, X. Ding, K. T. Kunnath, K. Zheng, R. N. Dave, Surface engineered excipients: III. Facilitating direct compaction tableting of binary blends containing fine cohesive poorly compactable APIs, International Journal of Pharmaceutics, 557, pp. 354-365, 2019). Besides, the use of well-flowing excipients, which tend to be coarser in size along with finer drug powders is likely to promote downstream segregation of APIs and excipients.

There are several approaches to improving flowability and bulk density of such blends such as dry granulation and wet granulation to name a few. Such technologies have proved to be successful only for specific drugs and drug loadings of an active pharmaceutical ingredient (API). In addition there are many disadvantages to using the current techniques of dry and wet granulation including but not limited to additional equipment required and added manufacturing expense due to the additional steps in these processes.

Granulation, the process of particle enlargement by agglomeration technique, is one of the most significant unit operations in the production of pharmaceutical dosage forms, mostly tablets and capsules. Granulation process transforms fine powders into free-flowing, dust-free granules that are easy to compress. Nevertheless, granulation poses numerous challenges due to high quality requirement of the formed granules in terms of content uniformity and physicochemical properties such as granule size, bulk density, porosity, hardness, moisture, compressibility, and the like together with physical and chemical stability of the drug. Typical granulation process can be divided into two types: wet granulation that utilizes a liquid in the process and dry granulation that requires no liquid. The type of process selection requires thorough knowledge of physicochemical properties of the drug, excipients, required flow and release properties as well as other properties.

Further, techniques such as wet granulation, which consists of multiple steps and use of liquids, are unsuitable for numerous APIs because of their tendency for degradation in presence of liquids or binders as well as heat during drying. Techniques such as dry granulation may also have certain limitations and may not work. For example, when the drug loading is outside the range between 20-60%. In addition, when more than one API or multiple component APIs are used, the tableting process becomes more complicated and has increased failures in making a tablet.

In the prior art technologies, silicas (silicon dioxide) are used in the pharmaceutical formulation (for example as glidants, as anti-caking agents, and as adsorbents) for potentially enhancing the blend flowability. For most such usage, the amount of silica typically ranges from 0.25 to 2.00 wt % of the blend. The lower values of this range are usually insufficient for flow enhancement for the blends with finer APIs and the higher values of this range may have adverse impacts such as uneven distribution of silica, potential separation of silica in the downstream processing, or variability of ensuing flow properties including reduction in the bulk density. In numerous prior art examples, it has been shows that direct addition of silica within the blend does not provide the required property enhancements, see for example; Improved blend and tablet properties of fine pharmaceutical powders via dry particle coating, Z. Huang, J. V. Scicolone, X. Han, R. N. Davé, (2015) International Journal of Pharmaceutics, Vol. 478 (2) p447-455.

The above cited prior art and others, for example, “Improved properties of fine active pharmaceutical ingredient powder blends and tablets at high drug loading via dry particle coating”; K. Kunnath, Z. Huang, L. Chen, K. Zheng, R. Davé, (2018), International Journal of Pharmaceutics, 543 (1-2), pp. 288-299 have addressed enhancement of flow properties of blends and in some cases, compaction properties of blends by dry coating the API powder with silica for API loadings ranging from 10 to 60 wt % and using adequate amounts of silica. However, as discussed herein, the above article did not consider either exceptionally low API loading or exceptionally low amounts of silica. Besides, for higher drug loading cases, the entire amount of API in the formulation had to be dry coated, hence requiring significant amount of processing and higher amounts of silica.

Indeed, current technologies require additional process steps in generating tablets that contain low amounts of API and/or have fine particle sizes and/or have mulitcomponents. Such additional steps generate quality issues due to reproducibility and increase manufacturing costs. Compaction properties such as tensile strength of the tablet suffer as agents are added to the pharmaceutical blend to increase flowability for better tablet processing. It is generally known and accepted in the art that as the flow properties increase for a pharmaceutical blend having fine particles or nanoparticles then the tensile strength of the tablet significantly decreases making the tablet process more difficult.

The use of flow promoting agents such as silica by itself does not lead to improved blend compaction properties. Examples are shown where dry blending of the similar ingredients does not lead to improved compaction properties as compared to the same ingredients that undergo wet granulation type processes that create intimate contact between microcrystalline cellulose (MCC) and silica. The addition of silica may reduce the free surface energy of the mixture because, in most cases, silica has lower surface energy than the excipient. However, the presence of silica can lead to inferior compaction properties since lower surface energy leads to weaker tablets. For example, in Fichtner, F., et al., “Effect of surface energy on powder compactability,” Pharmaceutical Research 25, 2750-2759 (2008), the article showed a decrease in tablet strength correlated to the decrease in powder surface energy at constant tablet porosities.

Thus, dry processing of an ordinary excipient with silica is not expected to lead to improved tablet compaction properties, even though the flow may be enhanced because of silica.

Overall, the prior efforts have attempted to disclose various aspects of better compacting excipients that include silica, surfactant, and other materials, they have not shown how dry processing can lead to better blends or better compacting individual excipients. Rather, it has been demonstrated that dry blending did not provide improved compaction properties, see for example, Chattoraj, S., et al., “Profoundly improving flow properties of a cohesive cellulose powder by surface coating with nano-silica through co-milling,” Journal of Pharmaceutical Sciences 100, 4943-4952 (2011); and Zhou, Q., et al., “Preparation and Characterization of Surface-Engineered Coarse Microcrystalline Cellulose Through Dry Coating with Silica Nanoparticles,” Journal of Pharmaceutical Sciences, 101:4258-4266 (2012). It was shown by these authors that dry coating of silica on fine (Avicel® 105), and coarse (Avicel 102) excipients may be achieved using many passes of a conical milling device, e.g., comil.

It was shown by the present inventors that dry coating of silica on fine (Avicel® 105), and coarse (Avicel 102) excipients may be achieved using one or two passes of a conical milling device, e.g., comil, if silica and excipient were pre-blended in a conventional mixer such as the V-blender prior to running through a comil. The resulting product from either approach was found to have enhanced flow. These dry coated excipients, however, produced weaker 100% MCC placebo tablets, although the tablet strength was found to be still acceptable for Avicel 105 as long as sufficiently high compaction force was used. However, this prior art did not consider or report tablet compaction using pharmaceutical blends of API and/or low loading API and/or dry coated excipients. It also did not examine the impact on the other solid dosage form production, such as filling of capsules and sachets.

Overwhelmingly, the prior art suggests that dry coating will lead to poorer compaction properties because it is likely to lead to reduced surface energy after dry coating (Sun, C., “Decoding Powder Tabletability: Roles of Particle Adhesion and Plasticity,” Journal of Adhesion Science and Technology, 25:483-499 (2011); Fichtner, et al. 2008; Etzler, F. M., et al., Tablet tensile strength: An adhesion science perspective. Journal of Adhesion Science and Technology 25, 501-519 (2011); and Han, X., et al., Passivation of high-surface-energy sites of milled ibuprofen crystals via dry coating for reduced cohesion and improved flowability. Journal of Pharmaceutical Sciences 102, 2282-2296 (2013)).

The blends of dry coated excipients with poorly flowing and poorly compacting APIs, particularly for extremely high or exceptionally low API loadings, are expected to have inadequate compaction properties required for high quality tablets, and formation of plug or densification via tapping required for capsule formation would also be adversely impacted. Likewise, filling sachets is also expected to be challenging for blends of poorly flowing and compacting APIs.

Some prior attempts are seen in US 2018/0055775A1 and US 10,751, 288B2. These documents focus on the improvements in the powder flowability and compressibility in blends where one of the excipient components was dry coated. However, for the binary blends, comprised of micronized and cohesive active pharmaceutical ingredient (API) with the surface engineered excipient, the lowest amount of dry coated component was 40 wt %. For that case, the amount of nano-sized fumed silica (Aerosil®) used in the formulation ranged from 0.5 to 1.00 wt %. Based on the results shown in these documents in which as the amount of dry coated component decreased, the flowability enhancements diminished. Therefore, reducing the dry coated component and hence the total amount of silica would be expected to continue to exhibit significant undesired decrease in level of flowability enhancements.

In Huang, Z., J. V. Scicolone, X. Han and R. N. Davé(2015), “Improved blend and tablet properties of fine pharmaceutical powders via dry particle coating,” International Journal of Pharmaceutics 478 (2) 447-455, multi-component blends of micronized acetaminophen (˜ 10 microns size) with either fine (˜ 20 microns) or conventional, coarse (˜ 100+microns) excipients was researched. The drug/API loads considered were 10, 30 and 60 wt % hence in the lowest component of dry coated components was 10 wt %, and the lowest amount of silica in the blend was 0.26 wt %. Unfortunately, for that embodiment, the flow function coefficient (FFC) enhancement due to coating of the API was not adequate, i.e., FFC of about 5, to promote direct compaction, capsule, and sachet filling with reduced segregation tendency.

The flowability of solids may be classified according to shear-testing based assessment of the flow function coefficient (FFC), which is the ratio of the consolidation pressure or stress (σ1) divided by the cohesive strength (fc) or flow function or flow factor (FF) values. It is generally accepted that very cohesive and non-flowing solids, granules, powders, and the like have a FFC less than 2. Cohesive is defined as having a FFC greater than 2 and less than 4. An easy flowing solids, granules, powders, and the like have a FFC greater than 4 and less than 10. And free flowing is defined as having a FFC greater than 10. (See, Andrew Jenike, Storage and flow of solids, Bulletin 123 of Utah Engineering Experiment Station, Vol. 53, No. 26, November 1964).

In Huang, Z., J. V. Scicolone, X. Han and R. N. Davé (2015), “Improved blend and tablet properties of fine pharmaceutical powders via dry particle coating,” International Journal of Pharmaceutics 478 (2) 447-455, multi-component blends of micronized acetaminophen (˜10 microns size) with either fine (˜20 microns) or conventional, coarse (˜100+microns) excipients was researched. The drug/API loads considered were 10, 30 and 60 wt % hence in the lowest component of dry coated components was 10 wt %, and the lowest amount of silica in the blend was 0.26 wt %. Unfortunately, for that embodiment, when the blend utilized fine excipients, the flow function coefficient (FFC) enhancement due to coating of the API was not adequate, i.e., FFC of about 5, to promote direct compaction, capsule, and sachet filling with reduced segregation tendency.

However, FFC improved to almost 7 for the embodiment where the dry coated component was at 30 wt %, and the amount of silica in the blend was 0.78 wt %. A FFC of over 8 was achieved for the embodiment where the dry coated component was at 60 wt %, and the amount of silica in the blend was 1.57 wt %. The best FFC enhancements occurred for the case where the API loading was 10 wt % but all the fine components including the API and excipients were dry coated, amounting to excessive burden to conduct dry coating and the amount of silica in the blend to be well above 1 wt %. These results suggested that the trend for flowability enhancements favor increasing of the dry coated component such as the API and as silica increased significantly to over 1 wt % of the blend. Discouragingly, these prior art results further show a reduction in silica amounts along with the percentage of dry coated API component will result in decreased FCC.

In Huang, Z., W. Xiong, K. Kunnath, S. Bhaumik, and R. N. Davé (2017), “Improving blend content uniformity via dry particle coating of micronized drug powders.” European Journal of Pharmaceutical Sciences 104:344-355, reported was a study of the binary blend which was comprised of the dry coated minor component in the amounts equivalent to 3, 5, or 10 weight percent of a cohesive API (with or without dry coating) and the Avicel PH102. The purpose of the work was to demonstrate if dry coating of the API would improve the drug content uniformity and not the flowability or processability of the blend. Huang et al. used a binary blend instead of more pharmaceutically relevant multi-component blends that offer additional challenges in processability.

In Kunnath, K., Z. Huang, L. Chen, K. Zheng, and R. Davé (2018). “Improved properties of fine active pharmaceutical ingredient powder blends and tablets at high drug loading via dry particle coating.” International Journal of Pharmaceutics 543 (1-2): 288-299, additional cases of APIs were considered to assess the repeatability of the prior-art publication that only included a single API test-case. Kunnath et al. 2018 investigated multi-component pharmaceutical powder blend at high loadings or 60 weight percent API loading for assessing blend's flowability and bulk density change due to the dry coated APIs with 1 wt % of either hydrophobic or hydrophilic silica. While the property enhancements reported were adequate, the paper focused only on high dry loading of 60 wt %, hence the dry coated component in the blend was high, at 60 wt %, and correspondingly, the silica was 0.6 wt % of the total blend.

In Kim, S., E. Bilgili and R. N. Davé (2021), “Impact of altered hydrophobicity and reduced agglomeration on dissolution of micronized poorly water-soluble drug powders after dry coating.” International Journal of Pharmaceutics 606 reported was the competition between the increase in the surface hydrophobicity and decrease in the agglomerate size on the dissolution rate of the poorly water-soluble API without forming a blend. Researched was the examination of properties of the API with and without dry coating, and hence the dry coated constituent was 100%, and the lowest silica amount was 0.28 wt %. Multicompetent APIs were not investigated as well as low loading APIs of less than 5 wt %. The prior research indicated that low loading and low use of silica would not be favored for direct tabulation, and other processing would be needed. Interestingly, this work pointed out that the flowability and natural agglomeration of fine powders are inversely corelated, implying improved flowability may also be associated with reduced agglomeration, which could be a quick indicator of the fine powder cohesivity.

Osorio and Muzzio (2013), “Effects of powder flow properties on capsule filling weight uniformity” Drug Development and Industrial Pharmacy 39 (9): 1464-1475, noted that for the optimal encapsulation, powder blends or single component powders must have the right flowability and bulk density. They concluded that “the better the flow, the higher the weight and the lower the variability for such as capsule filling system.” These properties become even more critical as the API loading is reduced as they affect the product's homogeneity. The paper also mentioned that while a single measure of flowability may be insufficient to discern various powder blends cases, overall, better flowability and bulk density ensure processability and product quality for capsules. However, this paper did not discuss reliable methods to enhance blend properties.

Thus, there remains a need in the art to address the significant problems faced by formulators in pharmaceutical, nutraceutical and other industries that deal with preparing blends of active ingredient mixed with other functional materials. For example, in pharmaceutical industry, this requires significant amount of development time, materials, and effort for developing blends to form tablet, capsule or sachet formulations that exhibit desirable properties such as flowability, bulk packing density, compressibility or compactability, blend content uniformity, as well as dissolution from the API powders, without having to resort to granulation, especially wet granulation that consists of multiple steps and use of liquids.

This problem is even more challenging for multicomponent API blends when the APIs are fine sized powders, ranging from 10 to 70 micron, usually further categorized as fine (10-15 micron) or semi-fine (30-50 micron) with low (less than about 5 wt %) active pharmaceutical ingredient (API) loading. While it is well known that when the blend includes flow aids such as fumed silica, the blend properties such as flowability can be improved. Unfortunately, the improvements by simply adding the silica during blending are not adequate as compared to those achieved if the silica was indeed dry coated, see for example Huang, Z., J. V. Scicolone, X. Han and R. N. Dave (2015), “Improved blend and tablet properties of fine pharmaceutical powders via dry particle coating,” International Journal of Pharmaceutics 478 (2) 447-455.

The problem remains challenging even for the extremely high drug loading formulations since a large amount of API powder must be dry coated. For example, a 100 Kg batch would require dry coating of 600 Kg fine API for a 60 wt % drug loaded formulation such as that discussed by Huang, Z., J. V. Scicolone, X. Han and R. N. Davé (2015), “Improved blend and tablet properties of fine pharmaceutical powders via dry particle coating,” International Journal of Pharmaceutics 478 (2) 447-455. Managing such large quantities per batch for a specialized processing step like dry coating also poses practical challenges. Therefore, it would be beneficial to be able to dry coat only a smaller portion of the batch and yet achieve adequate flow enhancements. For example, what if only 50 Kg of API were to be dry coated instead of 600 Kg in this example, yet achieve adequate flow for the final blend, where the total amount of silica used would be reduced by 10 times.

Current art does not provide such approaches and hence for nano-sized additives such as silica when dry coated on APIs, some of these properties could be improved, but require high silica amounts, e.g., over 1 wt % of the blend, and also require dry coating of exceptionally large amounts of material per batch while making tablet formation difficult. Further, it remains a challenge to achieve simultaneous improvements in all such properties while using lesser amounts of silica and when the APIs (or active ingredients in other industry applications) are fine.

### SUMMARY OF THE INVENTION

The present invention overcomes the drawbacks of previous attempts and provides additional benefits. Specifically the composition method concerns significant synergistic enhancement of desirable critical properties, such as improved bulk density, powder flow, and agglomerate size reduction, the powder or blend flowability, bulk packing density, blend uniformity, compactability, or drug dissolution, of blends of fine powders through solventless dry mechanical coating.

The current invention tackles the above problems of using hydrophobic coating agents through surprising collaborative synergy arising from a particular combination of hydrophobic and hydrophilic coating agents in multicomponent blend's bulk properties enhancements where flowability, bulk density, and deagglomeration enhancements match that of the hydrophobic dry coating agents while reaching significantly faster API dissolution or release profiles as if only hydrophilic dry coating agents were used. The collaborative enhancements from the combinations of hydrophobic and hydrophilic coating agents is highly advantageous because dry coating is not only providing solutions to an environmentally benign process that does not require the use of liquids and additional steps and such blends lead to simple dry mixing and compression to form tablets and capsules, but the combination of hydrophobic and hydrophilic dry coating agents alleviates the concerns around powder processability and API dissolution or release while the total weight percent of dry coating agents (for example, nano fumed silica) is less than 1 wt %.

The present inventors have found a synergic effect is obtained by coating a mixture of hydrophobic nano-fumed silica (such as microcrystalline cellulose (MCC) Aerosil® R972P) and hydrophilic nano-fumed silica (Aerosil® A200) simultaneously onto the surface of an active pharmaceutical ingredient (API) or drug, or even excipient powders. The examples shown herein illustrate various combinations in the weight percent ratios of hydrophobic dry coating agent to the hydrophilic dry coating agent where a preferred one is 1 to 3 as well as normalized surface area coverage ratios of 1 to 4. In other embodiments weight percentage ratios of hydrophobic dry coating agent to the hydrophilic dry coating agent of 5 to 1 are shown. It was found preferable when dry coating an API to use a dry coating ratio of 1 to 3 for increasing properties such as dissolution where more hydrophilic coating agent is used. In other embodiments, for example when dry coating an excipient, it was found preferable to use a dry coating ratio of 5 to 1 for increasing properties such as the flowability while minimizing the reduction in the tensile strength where less hydrophilic coating agent is used. Such dry coated embodiments exhibited surprisingly unexpected outcomes of improved bulk density, powder flow, and agglomerate size reduction for the milled and cohesive BCS I and BCS II classified drugs in the comparable or better range than the single component silica dry coating. In addition, remarkably surprising was the notable dissolution rate enhancement of poorly water-soluble BCS II class drug, even outperforming dry coated embodiments where only the pure hydrophilic silica (A200) was coated onto the milled BCS II API. Such an outcome is rather surprising because an opposite result was expected. Similarly, unexpected enhancements of three desirable properties; flow, bulk density, and compaction of a fine excipient were achieved by coating a mix of hydrophobic nano-fumed silica (Aerosil® R972P) and hydrophilic nano-fumed silica (Aerosil® A200) simultaneously onto the surface of the excipient powders.

In another aspect, the present inventors found significant unexpected enhancement of properties of individual fine drug powders, or their blends, obtained by coating a mix of hydrophobic and hydrophilic coating agents onto the surface of the drug (or excipient) powders that are cohesive or very cohesive Enhanced dissolution of fine drug powders with binary coatings as compared to individual coatings had surprisingly positive results such as certain addition of a hydrophobic coating agent leads to faster dissolution as compared with even complete hydrophilic coating, or all hydrophobic coating or no coating at all. The present inventors found better flowability of fine drug or excipient powders with binary coatings as compared to individual coatings, as addition of small amount of hydrophobic coating agent led to such enhancement. In addition it was found that higher tablet tensile strength was achievable when small amounts of hydrophobic coating agent was added, as compared with no coating. Such coatings are expected to have advantages for blend containing individual component coated with binary coating agents.

The present invention provides for formulations in pharmaceutical industry that simplify the development without requiring multi-step processing which could require liquids, binders, drying, and the like, leading to direct compaction manufacturing of drug products with increased API dissolution and or release rate. Pharmaceutical and other industries that require improved processability of fine powder blends that are typically cohesive and have poor processability and poor solubility benefit from the present invention.

In one aspect, a method of using binary coating agents in powder coating for cohesive or very cohesive particles, comprises taking one or more powder components having a flow function coefficient (FFC) under 4 FFC prior to any surface modification or treatment, wherein at least one of the components is an active pharmaceutical ingredient (API) or an excipient; dry coating by a binary dry coating with both a hydrophobic dry coating agent and a hydrophilic dry coating agent on a surface of the API or the excipient or both the API and the excipient for forming a dry coated component, using for the hydrophobic dry coating agent and the hydrophilic dry coating agent a weight percentage ratio or a ratio of the normalized surface area coverage (SAC) for the dry coated component, and providing by the binary dry coating flowability, bulk density, deagglomeration, and dissolution that are substantially different for the dry coated component and/or a blend containing the dry coated component as compared to using the hydrophobic or the hydrophilic dry coating agents alone or no dry coating agents used.

In another aspect, by combining hydrophobic and hydrophilic nano-fumed silica in a specific ratio to dry coat an individual powder or a component within a multi-component blend (including but not limited to API and disintegrant), wt % of silica with respect to API or excipient is about 2.31, and as low as 0.01% for multi-component blends were found effective promoting not just direct compression tableting but also tailoring powder processability and API release rate in more targeted manner, which was not evident earlier in the art.

In yet another aspect, a material-sparing dry coating method was adopted using a high-intensity vibrational mixer to dry-coat the fine and cohesive host powders. The host powder was either individual cohesive and fine pharmaceutical powder or one of the components (either minor or major) within a multi-component blend that contains more than three components. The host powder was dry coated with a mixture of Aerosil® R972P (or any hydrophilic nano-fumed silica/nano-sized pharma grade dry coating agents) and Aerosil® 200 (or any hydrophilic nano-fumed silica/nano-sized pharma grade dry coating agents) in the weight percent ratio of 1 to 3 or normalized surface area coverage ratio of 1 to 4, resulting the total wt % of silica in the multi-component blends in the range of 2.31 to 0.01 wt %.

The dry coating device depending on the implementation could be any mechanical mixer, for example, tumbler mixer, Mechan fusion device, MAIC, v-blender, tote mixer, twin-screw mixer, and the like, or any mechanical impact device for example, Comil, fluidized energy mil, ball mill, and the like. The result of dry coating quality is independent of the addition order of the hydrophilic and hydrophobic nano-fumed silica. However, suppose soft and wax-like substances like Magnesium Stearate (MgSt) may be used as the dry coating materials. In that case, the addition order affects the final product quality and functionality depending on the mechanical mixer type used for dry coating. Thus, depending on the soft and easily spreadable dry coating materials' hydrophobicity, the addition order needs to be kept consistent. For the case of hydrophobic, may be added first. For the case of hydrophilic, may be added second. Preferably the hydrophobic and hydrophilic coating agents are added simultaneously as further discussed herein. Hydrophobic silica effectively reduces the fine powders' interparticle cohesion force as it tends to have lower surface energy than hydrophilic silica. Consequently, the main contribution of hydrophobic silica is bulk flowability and bulk density enhancement with reduced agglomeration tendency.

While hydrophilic silica can also function to reduce the interparticle cohesion force of the fine powders as given its nano size, its effectiveness is less than that of hydrophobic silica due to its higher surface energy. However, given its significantly greater polarity and thus hydrophilicity, the primary function of hydrophilic silica is to aid the dissolution and, thus, the release of the API from the final product, such as a tablet, capsule, or sachet. The selection of dry coating agents, including not but not limited to nano-fumed silica, is based on the surface energy and size differences from the fine powders (which could be either APIs or excipients). The guest or dry coating agents with greater size and surface energy differences (dry coating agents should have smaller size and lower surface energy than the host powders) from the host powders are the preferred selection.

This present disclosed method and formulation is highly advantageous because dry coating is an environmentally benign process that does not require use of liquids and additional steps and such blends lead to simple dry mixing to form compressed tablets, filled capsules and sachets. Further, the disclosed formulation and method reduce the need for dry coating the majority or the entirety of ingredients, hence reducing the processing burden. In one aspect, in a single component or multicomponent API blend having one or more than three components, and preferably at least 5 components, one or more components together as low as 1-5 wt % were dry coated keeping silica concentration in the blend in the range of 0.116 to 0.007 wt %.

The present invention utilizes a relatively low amount of silica dry coated (and not wet granulation or dry granulation techniques) on a minority component or majority component that results in an API blend of surprisingly high FFC values as well as unexpected high tensile strength and dissolution properties. A “minority” component for the purposes of this specification is defined as for a binary or at least a two component API blend the component that has less than 50% by weight volume, or for a single constituent, a minority fraction of material selected and processed for dry coating. In blends that have more than two API components, the minority component is the API with the least amount of weight per volume or a part thereof. Prior-art examples show that typically the majority component would need to be dry coated and not the minority component, or at the least, the entire minority component, equal to or exceeding 10 wt %. An average FFC calculated for the blend constituents including the dry coated minority component showed an expected low FFC.

However as shown herein, a high FFC resulted when the minority component was coated. This surprising result also occurred when the excipient was dry coated as a minority component instead of the API. It is within the scope of the invention to dry coat the minority component whether it is an API, an excipient, or dry coating both the API and the excipient together so long as it is a minority component.

Depending on the implementation, the APIs or excipients may be different or all the same at different concentrations. The current prior art failed approaches to enhance critical blend properties involve employing dry granulation or wet granulation that are multi-step processes, or use of excessive amounts of silica additives to promote just one or two properties. In contrast, the novel formulation taught by this invention leads to surprising outcomes for simultaneous enhancements of all such desirable properties, facilitating formulation design, product manufacturing, product quality, and patient experience. Such advantages are also expected in other applications involving the use of fine constituents.

Dry coating of a minor component in a blend, including a minor fraction of the component if it is not a minor component, improves upon these deficiencies of the prior art and surprisingly, it is found that dry coating does not have detrimental effect on tablet strength of blends of cohesive APIs at low drug loadings (less than 5 wt % such as 1, 3 or 5 wt %) that have multicomponent APIs, and APIs with fine particle size. For purposes of this disclosure fine particle size is defined as active ingredients, ranging from 10 to 70 microns, and further categorized as fine (10-15 micron) or semi-fine (30-50 micron) with low (less than about 5 wt %) active pharmaceutical ingredient (API) loading.

The use of the silica in the present disclosure has a simplified manufacturing scheme and minimal use of silica or other flow promoting agents. Accordingly, dry coated excipients can be produced by a manufacturing process that does not require the use of any liquids via simultaneous milling and dry coating. Dry coated excipients can also advantageously be used to form cohesive API blends by direct compression or roller compaction without the need for wet granulation. In addition, such practice will simplify the manufacturing processes for filling capsules and sachets.

The dry coating in the current invention addresses the use of minor components in the more challenging cases of multi-component blends, where the dry coated component is at the most at 5 wt %, and more remarkably, silica concentration in the entire blend is in the range of 0.116 to 0.007 wt %. Even at this low silica concentration, it presents remarkable flowability and density improvement, beyond the flowability range described in the prior patents.

The silica particles used in the present invention may be of any type, such as hydrophobic treated silica or fumed silica. Examples of suitable silicas include, but are not limited to, Aerosil R972 silica (Evonik), CAB-O-SIL EH-5 silica (Cabot), CAB-O-SIL M-5P silica (Cabot), CAB-O-SIL M-5DP silica (Cabot), AEROS IL® 200 Pharma (Evonik), AEROSIL® 300 Pharma (Evonik), AEROPERL® 300 Pharma (Evonik), OX-50 silica (Evonik), and TS530 silica (Cabot). In general, preferred are the fumed amorphous silicas with a specific surface area of greater than about 100 m²/g. Hydrophobic silica may be obtained by hydrophobic treatment. In one embodiment, the hydrophobic treatment of hydrophilic silica may be accomplished by treating the hydrophilic silica with dichlorodimethanolsilane. Any other suitable methods known to a skilled person that are capable of modifying silica to hydrophobic silica may be used. Hydrophobic silica, in addition to improving flow and bulk density properties, may aid in improved dispersion of the coated API and greatly reducing the size of agglomerated API particles, aiding in their faster release.

In some aspects of the invention, the silica comprises hydrophilic silica having a specific surface area ranging from about 175 m²/g to about 300 m²/g.

In some aspects of the invention, the silica comprises a functionalized hydrophobic having a specific surface area ranging from about 90 m²/g to about 130 m²/g.

In some aspects of the invention, the API or the selected minor component of the blend has a bulk density ranging from about 0.05 g/mL to 0.5 g/mL and a flow function coefficient (FFC) ranging from about 1.0 to 3.5.

In some aspects of the invention, the API or the selected minor component of the blend is present in an amount ranging from about 0.1 wt % to about less than 5%, based on the total weight of the pharmaceutical blend.

In some aspects of the invention, a flow function coefficient (FFC) of the dry coated pharmaceutical ranges from 3 to 30.

In some aspects of the invention, the bulk density of the pharmaceutical blend ranges from about 0.2 g/mL to about 0.99 g/mL.

In some aspects of the invention, the flowability of the pharmaceutical blend ranges from about 2 to about 30.

In some aspects of the invention, the silica has a particles size ranging from about 10 to about 50 nanometers and is present in an amount of about 0.01 wt % to about 1 wt %, based on the total weight of the pharmaceutical.

In some aspects of the invention, a pharmaceutical tablet is made from the blend, the tablet having a porosity ranging from about 0.05 to about 0.35 and a tensile strength ranging from about 1 MPa to about 10 MPa.

In some aspects of the invention, a method of forming a pharmaceutical tablet includes compressing a multicomponent blend of an API or the selected minor component of the blend that is dry coated with a silica to directly compress the blend into a pharmaceutical tablet, wherein the method does not include a wet granulation step.

In some aspects of the invention, a pharmaceutical capsule is made from the blend, the capsule of size 1 is filled with blend weight ranging from about 250 mg to about 400 mg and its weight variation under 5%.

In some aspects of the invention, a method of filling a pharmaceutical capsule includes using a dosator to compress a plug of a multicomponent blend of an API that is dry coated with a silica, wherein the method does not include a wet granulation step.

In another aspect of the invention, using a material sparing high-intensity vibrational mixer, one of the minor components within a multi-component blend which contains more than three components was dry coated with the 2.31 to 1 wt % of either Aerosil R972P or Aerosil 200, hence resulting in the silica amount of only 0.023 to 0.01 wt % in the multi-component blend. The coating materials were selected based on the surface energy differentials in the API and silica.

Depending on the implementation of the current invention, finer sized silica is preferred as defined herein. The flowability, bulk density, API release rate, and compaction strength were remarkably improved unexpectedly even at such a low silica concentration. For example, for cohesive and fine multi-components (three to five components), the flowability improved up to 100%, resulting in the flow regime shift from cohesive to easy flowing. Likewise, the bulk density was improved, and remarkably, the compaction properties also surprisingly improved even in presence of silica.

Most impressively, using the current invention for single or multiple components blend, the drug dissolution rate was significantly improved as compared to the untreated five components blend. Current approaches involve employing dry or wet granulation and multi-step processes to enhance one or two of the desirable blend properties such as blend flowability, bulk packing density, blend uniformity, compaction, or drug dissolution. The challenges are more significant for the blends that have fine powders. Further, current approaches may also require use of significant amounts of additives to promote one or more properties.

In contrast, the novel formulation of the current invention and approach leads to surprising synergistic results for simultaneous enhancements of all such desirable properties, facilitating formulation design, product manufacturing, product quality, and patient experience. Such advantages are also expected in other applications involving the use of fine constituents. The flexibility and variations in the invention arise from the synergy during blending as the additives such as nano silica are coated via any method on to one of the fine powder ingredients, including the smaller fraction of the same ingredient.

In some aspects of the invention, dry coating on a reduced multicomponent active pharmaceutical ingredient (API) agglomeration is disclosed herein. Fine cohesive API agglomeration through dry coating was shown to enhance uniformity and processability of multi-component blends at low (1, 3, or 5 wt %) API loadings. Dry coating of fine milled ibuprofen (˜10 um) was systematically assessed with two distinct types/amounts of silica. Dry coated ibuprofen powders exhibited dramatic agglomeration reduction, decreased cohesion, unconfined yield strength, and improved flowability attributed to nano-scale surface morphology imparted by silica coating. Blends exhibited profound enhancement in flowability/bulk density at above low API loadings. Hydrophobic silica coating improved drug dissolution rate without appreciably reducing tablet tensile strength. Dry coating all three low drug loading APIs improved blend uniformity and tablet dissolution due to agglomerate size reduction. Blend flowability, bulk density, and blend processability were improved without adverse impact on tablet compaction. Although these examples illustrate objects and advantages of the present invention for a poorly water-soluble drug ibuprofen, which is a BCS (biopharmaceutical classification system) Class II drug, it is applicable to other such cases, including fenofibrate, griseofulvin, itraconazole, naproxen, sulfamethoxazole, phenylbutazone, azodicarbonamide, danazol, albendazole, nifedipine, cilostazol, ketoconazole, budesonide, loviride, glimepiride, biphenyl dimethyl dicarboxylate, digitoxin, paclitaxel, predinisolone acetate, hydrocortisone acetate and any suitable mixtures thereof.

The above objects and advantages are met by the presently disclosed method and apparatus. In addition, the above and yet other objects and advantages of the present invention will become apparent from the hereinafter set forth Brief Description of the Drawings, Detailed Description of the Invention, and claims appended herewith.

These features and other features are described and shown in the following drawings and detailed description. Furthermore, any combination and/or permutation of the embodiments are envisioned.

Again, other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIGS. 1A-1F are, respectively, scanning electron microscope (SEM) images for fixed silica weight % dry coated samples for: (a) uncoated Ibuprofen (Ibu) 10, (b) A200-2.31 wt %, (c) R972P-0.58 wt %-A200-1.73 wt %, (d) R972P-1.16 wt %-A200-1.16 wt %, (e) R972P-1.73 wt %-A200-0.58 wt %, and (f) R972P-2.31 wt %.

FIG. 2 illustrates a chart showing Dissolution rates evaluated for the uncoated and dry coated milled and cohesive Ibuprofen (Ibu) powders using USP IV method with de-ionized water as the dissolution medium.

FIGS. 3A-3F illustrate SEM images for fixed SAC & dry coated samples: (a) uncoated Ibu10, (b) A200-50% SAC, (c) R972P-5% SAC-A200-45% SAC, (d) R972P-7.8% SAC-A200-42.2% SAC, (e) R972P-10% SAC-A200-40% SAC, and (f) R972P-50% SAC.

FIG. 4 is a chart illustrating Dissolution rates evaluated for the uncoated and dry coated milled and cohesive Ibu powders using USP IV method with de-ionized water as the dissolution medium.

FIGS. 5A-5G are SEM images for fixed SAC % dry coated samples, respectively: (a) uncoated mAPAP, (b) R972P-100% SAC, (c) A200-100% SAC, (d) R972P-50% SAC, (e) A200-50% SAC, (f) R972P-20% SAC-A200-80% SAC, and (g) R972P-10% SAC-A200-40% SAC.

FIGS. 6A-6B are charts illustrating API release rate from 200 mg, 12.7 mm tablets which are comprised of ternary blend with 40% of API loading by weight for (a) 100% SAC cases with varying dry coating formulation, and (b) 50% SAC cases with varying dry coating formulations.

FIGS. 7A-7D illustrate SEM images of, respectively: (a) uncoated microcrystalline cellulose (MCC) Avicel® 105; (b) 1 wt % A200 coated Avicel® 105; (c) 1 wt % R972 coated Avicel® 105; (d) 0.32 wt % A200 and 0.54 wt % R972 coated Avicel® 105.

FIG. 8 is a bar graph illustrating a plot of FFC for powder in Example 10.

FIG. 9 is a bar graph illustrating a plot of bulk density for powder in Example 10.

FIG. 10 is a bar graph illustrating a plot of tensile strength for powder in Example 10.

FIGS. 11A-11C are SEM images of respectively: (a) uncoated microcrystalline cellulose (MCC) excipient Pharmacel® 101; (b) 1 wt % A200 coated Pharmacel® 101; (c) 0.34 wt % A200 and 0.55 wt % R972 coated Pharmacel® 101.

FIG. 12 illustrates a plot of FFC for blends in Example 11.

FIG. 13 illustrates a plot of bulk density for blends in Example 11.

FIG. 14 illustrates a plot of tensile strength for blends in Example 11.

FIG. 15 illustrates a table showing dry coating formulation details used for the study.

FIG. 16 illustrates a table showing characteristic particle size distribution (PSD) for the uncoated and dry coated milled Ibu measured in two different methods to assess primary and agglomerated PSD.

FIG. 17 illustrates a table showing bulk properties assessments on the milled Ibu before and after the dry coating using FT4. Shear testing or flowability was assessed under 3.0 kPa consolidation pressure.

FIG. 18 illustrates a table showing properties of testing liquids for the modified Washburn testing.

FIG. 19 illustrates a table showing liquid penetration method: surface contact angle results for fixed silica weight % cases.

FIG. 20 illustrates a table showing dry coating formulation details used for the study.

FIG. 21 illustrates a table showing Characteristic particle size distribution (PSD) for the uncoated and dry coated milled Ibu measured in two different methods to assess primary and agglomerated PSD.

FIG. 22 illustrates a table showing bulk properties assessments on the milled Ibu before and after the dry coating using FT4. Shear testing or flowability was assessed under 3.0 kPa consolidation pressure.

FIG. 23 illustrates a table showing liquid penetration method: surface contact angle results for fixed SAC % cases.

FIG. 24 illustrates a table showing dry coating formulation details used for the study.

FIG. 25 illustrates a table showing characteristic particle size distribution (PSD) for the uncoated and dry coated milled Ibu measured in two different methods to assess primary and agglomerated PSD.

FIG. 26 illustrates a table showing bulk properties assessments on the milled Ibu before and after the dry coating using FT4. Shear testing or flowability was assessed under 3.0 kPa consolidation pressure.

FIG. 27 illustrates a table showing formulation of example 10.1 to example 10.8.

FIG. 28 illustrates a table showing formulation of example 14.1 to example 14.8.

FIG. 29 illustrates a table showing formulation details for fixed silica weight % dry coated Ibu10 cases and fixed surface area coverage % of dry coated Ibu10.

FIG. 30 illustrates a table showing properties of testing liquids for the modified Washburn testing.

FIG. 31 illustrates a table showing API particle size evaluation at two different dispersion methods for fixed silica weight % cases.

FIG. 32 illustrates a table showing FT4 test results for fixed silica weight % cases.

FIG. 33 illustrates a table showing Liquid penetration method: surface contact angle results for fixed silica weight % cases.

FIG. 34 illustrates a table showing Area under the dissolution curve (AUC) for fixed silica weight % cases.

FIG. 35 illustrates a table showing API particle size evaluation at two different dispersion methods for fixed SAC cases.

FIG. 36 illustrates a table showing FT4 test results for fixed SAC % cases.

FIG. 37 illustrates a table showing Liquid penetration method: surface contact angle results for fixed SAC % cases.

FIG. 38 illustrates a table showing Area under the dissolution curve (AUC) for fixed SAC % cases.

FIGS. 39A-39F illustrates SEM images for fixed silica weight & dry coated samples, respectively: (a) uncoated Ibu10; (b) A200-2.31 wt %; (c) R972P-0.58 wt %-A200-1.73 wt %; (d) R972P-1.16 wt %-A200-1.16 wt %; (e) R972P-1.73 wt %-A200-0.58 wt %; (f) R972P-2.31 wt %.

FIG. 40 illustrates dissolution profiles from UPS IV testing at fixed silica weight %. De-ionized water was used as the dissolution medium.

FIGS. 41A-41F illustrate SEM images for fixed SAC % dry coated samples, respectively: (a) uncoated Ibu10; (b) A200-50% SAC; (c) R972P-5% SAC-A200-45% SAC; (d) R972P-7. 8% SAC-A200-42.2% SAC; (e) R972P-10% SAC-A200-40% SAC; (f) R972P-50% SAC.

FIG. 42 illustrates Flow function coefficient for both cases: fixed silica weight % and fixed SAC %.

FIG. 43 illustrates a relationship between powder porosity and SAC %.

FIG. 44 illustrates dissolution profiles from UPS IV testing at fixed SAC %. De-ionized water was used as the dissolution medium.

FIG. 45 illustrates a table showing bulk, true powder densities and porosities of each sample listed.

FIGS. 46A-46B illustrates plot graphs showing the correlation between normalized effective radius of voids to geometric packing factor and to surface contact angle, respectively, for FIG. 46A fixed silica weight %; and FIG. 46B fixed SAC %.

### DETAILED DESCRIPTION

For purposes of this description, range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 and the like, as well as individual numbers within that range, for example, 1, 2, 2.3, 3, 4, 5, 5.7 and 6. This applies regardless of the breadth of the range.

Unless noted otherwise, particle size distribution provided herein is volume-based particle size distribution was measured by a laser diffraction particle analyzer (Rodos/Helos system, Sympatec, NJ). Size statistics in terms of d₁₀, d₅₀ and d₉₀ are reported, which are the values of the particle diameter at 10%, 50% and 90% respectively in the cumulative volumetric particle size distribution. In the Rodos/Helos system, the Rodos device works by venturi principle to disperse the powder, and the Helos unit uses laser diffraction principles of Fraunhofer Enhanced Evaluation (FREE) and Mie Extended Evaluation (MIEE) theories of light scattering to determine the particle size. Size statistics of d₁₀, d₅₀ and d₉₀ at dispersion pressure of 0.1 bar are reported utilizing the FREE theory, the details for which can be found in Han et al. “Passivation of high surface energy sites of milled ibuprofen crystals via dry coating,” Journal of Pharmaceutical Sciences, Vol. 102 (7), 1-15 (2013) and Jallo et al. “Improvement of flow and bulk density of pharmaceutical powders using surface modification,” International Journal of Pharmaceutics, 423 213-225 (2012).

Unless otherwise noted, bulk density and flowability, i.e., flow function coefficient (FFC), as provided herein is obtained using a Freeman FT4 powder tester (Freeman Technologies Ltd., Worcestershire, UK), where the bulk density and the flow function coefficient (FFC) were defined as the ratio of consolidation stress to the unconfined yield stress. The FFC was evaluated from a shear test under the consolidation pressure of 3 kPa on the recommendation of 205 ASTM standard for powders having low density (Emery et al., 2009; Emery, E., Oliver, J., Pugsley, T., Sharma, J., Zhou, J., 2009, “Flowability of moist 688 pharmaceutical powders,” Powder Technol. 189, 689 409-415.) and the range normally used for pharmaceutical powders. Detailed procedures for both may be found in Freeman et al., Measuring the flow properties of consolidated, conditioned, and aerated powders —A comparative study using a powder rheometer and a rotational shear cell. Powder Technology 174, 25-33 (2007) and Huang et al., Flow and bulk density enhancements of pharmaceutical powders using a conical screen mill: A continuous dry coating device. Chemical Engineering Science 125, 209-224 (2015).

Though FFC is the flowability measure used herein, one of ordinary skill in the art would recognize that other flow indices can be used, such as angle of repose, Carr index, Hausner ratio, flow through orifice testing device, and others. In addition an agglomerate ratio may provide one such measure that requires lesser samples. FFC values reported here are obtained using the FT4 shear tester are pre-consolidation of 3kPA, unless otherwise stated. However, one of ordinary skill in the art would be able to use a suitable ring shear tester or equivalent at a suitable pre-compaction pressure, for example a novel material sparing powder strength tester device called the SpinTester-X (by Material Flow Solutions, Inc.), to determine FFC at low pre-consolidation in a manner comparable to FT4. A classification of powder flow behavior based on FFC is defined by Schulze, D., Powders, and Bulk Solids. Springer (2008) according to the FFC value: “FFC<1-not flowing, 1<FFC<2-very cohesive, 2<FFC<4-cohesive, 4<FFC<10-easy flowing, and FCC>10-free-flowing”.

Bulk density was measured through a standard FT4 testing procedure that first conditions the powder to yield very repeatable results for the bulk density as discussed in the detailed procedures of Freeman, et al. mentioned above. Prior to powder characterization the powder was conditioned to remove stress or excess air from the powder bed by passing a conditioning blade through the powder bed. This process will be referred to as the conditioning cycle. The conditioned bulk density was measured by loading the powder samples into the 25 ml split vessel above the minimum fill level. After a conditioning cycle was performed, the vessel was split to remove the top portion of the powder; the density was determined from the mass of the remaining powder in the 25 ml vessel.

The powder mixing process is a common yet critical part of numerous industry sectors including, agriculture, food, cement, plastics, and pharmaceutical. The mixedness or homogeneity of powder blends is one of the key factors affecting the final product quality, especially for pharmaceutical products, as it is a major regulatory requirement for the final dosage. For active pharmaceutical ingredients (API) loading at or below 5 weight percent, achieving the targeted content uniformity is challenging. In particular, even if mixing were ideal, the lowest possible coefficient of variation (Cv), a standard measure for the quality of mixing, is inversely proportional to the drug dose and proportional to the API particle size.

Hence, available models to estimate Cv recommend using finer drug powders for improving blend homogeneity as drug dose and loading are reduced. Unfortunately, these models assume ideal mixing, which is a non-trivial operation for fine API powders due to their cohesive nature, and further, they do not explicitly account for API agglomeration, hence model predicted Cv values are not achievable.

Classical approaches to achieving excellent drug content uniformity (CU), which may be assessed via Cv, include using wet granulation, and in some cases using ordered mixtures. For the latter, cohesive and finely milled API particles are deposited during mixing onto the surface of the coarse excipients that are one or more magnitudes larger in size than the API. The advantage of wet granulation is that it can be used for both high and low drug loaded blends because it could improve the flowability due to the size enlargement or enhance drug content uniformity due to better API dispersion within the granules that naturally counters the tendency for fine API particle agglomeration. However, the process is energy and resource-exhaustive due to the number of steps required and the need to use water and other solvents that could impair API stability. For exceptionally low drug loadings, ordered mixtures are also an option where fine API particles stay attached due to their interparticle cohesion force with coarse excipient particles. As a result, API agglomeration problem is reduced, and uniform distribution of the API could be achieved along with improved flowability of the powder blend without being adversely impacted by fine API particles. Potential shortcomings of this approach include, limited drug loading, the need for using excipients that have narrow particle size distribution (PSD), specific mixing requirements, and a limited range of drug loadings. For example, the use of specialized higher intensity mixing devices and preferred types of excipients may be needed to attain desirable CU at exceptionally low drug loadings. Such difficulties in uniformly coating micro-sized APIs on carrier particles were also confirmed through an energy-based stick-bounce model.

The impregnation method has been used recently due to its capability of achieving good uniformity for ultra-low API loading where the API is deposited on porous non-dissolving and inert excipients. This approach could naturally provide excellent CU, since, in theory, the API is distributed throughout a larger, well flowing porous carrier particle. However, for commercial usage where avoiding downstream segregation could be challenging, as it would require excellent PSD control through milling or using a rather narrow sieve-cut size range. Other challenges could be the selection of suitable solvents that result in low viscosity API solutions, solvent recovery for reduced environmental footprint, and effective drying for complete removal of the solvents. Recent work pointed to additional issues such as slow and/or incomplete drug release and reduced storage stability in cases where the drug could attain nano-confinement induced amorphous state.

An alternate approach to encounter poor processability of fine APIs is the dry coating, a solvent-less method to reduce the cohesion of fine powders leading to improved bulk powder properties such as, but not limited to, flowability, packing density, fluidization, and dissolution. A significant advantage of dry coating is in enhancing powder blend properties such as flowability, bulk density, dissolution rate, as well as promoting direct compression tableting along with improved tablet properties at high drug loadings, even when only one of the constituents is dry coated. Most relevant to the current paper, several studies have also examined the impact of dry coating on reduced agglomeration, including subsequent impact on mixture uniformity at low API loading. For example, a model fine-sized (˜10 um) water-soluble API, micronized acetaminophen (mAPAP), was used at API loadings of 3, 5 and 10 wt % in binary blends with Avicel Ph-102 (˜120 um) to examine the influence of dry coating with a fixed amount (1 wt %) of hydrophobic silica R972P on the blend CU. Owing to the disparate sizes of the API and excipient, the condition where achieving uniformity would be challenging, and the effects of mixing time and the dry coating on the blend uniformity were explored.

However, the impact of dry coating on flowability and bulk density of the blends to promote direct compression tableting was not examined, nor the effect of diverse types and amounts of silica, as well as their impact on tablet dissolution was investigated, presumably because the API was readily water soluble. A major shortcoming was the assessment of agglomerate size estimated through sieving which provided limited resolution. A more recent study examined the impact of silica type and amounts on dissolution to better understand the combined effect of reduced agglomeration and altered surface hydrophobicity by considering poorly water-soluble APIs. Although it considered multiple methods for better assessing the size of agglomerates, it did not consider forming blends or tablets.

Dry coating with distinct types and amounts of silica may influence the API agglomerate sizes and consequently, the blend properties including CU at lower drug loadings as compared to the work by Huang et al (2017). It would be also interesting to examine the influence of altered surface hydrophobicity as well as agglomeration on dissolution from tablets. In addition, the impact of dry coating could be assessed on the blend flowability and bulk density that can improve downstream processability.

Although the examples and prior art discuss challenges in powder handling and powder-based solid dosage products concern pharmaceutical products, the current invention can be beneficially applied to wide ranges of powder based applications, ranging from food, agrochemical, specialty chemical, energy, metal, and cement industries, which require well flowing (FFC over 5 or 6) and well compacting (bulk density over 0.45 g/mL) powders (“Powder Flow Properties” by Kerry Johanson, Encapsulated and Powdered Foods, (2005) CRC Press, 9780429120152; Diederich, P., Mouret, M., Ryck, A. , Ponchon, F., EScadeillas, G. “The nature of limestone filler and self-consolidating feasibility-Relationships between physical, chemical and mineralogical properties of fillers and the flow at different states, from powder to cement-based suspension.” Powder Technology. 218:90-101).

To the knowledge of the present inventors, there are no available reports that investigated the changes in both the host (API) powder properties and dissolution rate by employing a mixture of fumed silica as the flow-agent. Instead, previous studies have evaluated the impact of shear stress or concentration of hydrophobic or hydrophilic fumed silica concentration on API release rate from the tablet blends (Pingali et al. 2011); (Pingali and Mendez 2014); (Huang et al. 2015). Thus, the current invention is designed to analyze the impact on the flowability, bulk density, surface morphology, and dissolution rates when the mixture of two coating materials varying in their hydrophilicity were employed as the dry coating agent. Some of these studies have utilized Magnesium Stearate, MgSt, as the hydrophobic coating material (Swaminathan and Kildsig 2002); (Pingali et al. 2011). MgSt, a commonly added lubricant in powder blends, is a wax-like substance that can be easily transferred onto the surface of other particles, especially API particles, during the mixing process as it can coat the surface of host particle like a film under shear strain (Pingali et al. 2011). Being a hydrophobic material, transferred MgSt onto the surface of the API particles could affect the particle's dissolution rate (Uzunović and Vranić 2007); (Pingali et al. 2011); (Pingali and Mendez 2014). Besides, the tensile strength of the tablet could also be adversely affected because the hydrophobic nature of MgSt hinders the interparticle bonding between the tablet components (Patel, Kaushal, and Bansal 2006); (Gohel and Jogani 2005). Therefore, when MgSt is included in the component mixtures, the mixing intensity and the order of addition of such glidants significantly impact bulk properties (Pingali et al. 2011); (Pingali and Mendez 2014). As the result, a typical powder blend does not contain more than 1% by weight of MgSt in the composition. Thus, considering these limitations, the present inventors employed a combination of hydrophobic-hydrophilic nano sized silica mixture, instead of an MgSt and hydrophilic silica mixture for dry coating, as the nano sized fumed silica particles does not deform nor cause filing coating under shear strains like MgSt particles.

The reported study by Kim et al., 2021 showed that for the micronized ibuprofen (10 and 20 μm, in their d₅₀, respectively), both the agglomerate size and surface hydrophobicity change after dry coating impacts the dissolution rate. The general conclusion was that whereas hydrophobic silica leads to greater agglomerate size reduction, hydrophilic silica is better for hydrophobicity reduction. As an extension of the finding of Kim et al., 2021, the current experimental setup was designed to investigate the possibility of enhancing the API release rate by inducing the synergistic effect through a judicious combination of hydrophilic and hydrophobic silicas. Hence, the current study was initiated based on two hypotheses in mind. (1) synergistic impacts from the silica mixture dry coating are visible when the dry coating effectiveness is guaranteed; (2) discrete and effective dry coating by the silica mixture that forms the minimum number of silica agglomerates on the surface of the host particle will highlight the synergistic impact even further. To capture shifts in the agglomerate size due to dry coating more clearly, used fluidized energy milled ibuprofen (Ibu), which has d₅₀ of 10 to 13 μm. This milled Ibu was categorized as Ibu10.

Again, the following materials and methods and examples are merely given as illustrations of the principles of the invention. The invention is not so limited to the following examples. Instead the examples are given to illustrate the principles of the invention in certain aspects. Various deviations from the examples given herein are possible.

### 2.0 Materials and Method

### Materials

Through fluidized energy milling, prepared 10 μm of ibuprofen powders by milling as received ibuprofen 50, which was sourced from BASF (South Bishop, Texas 78343). Given the size and low surface (Garg energy 2015), Aerosil 200 (nano-sized hydrophilic fumed silica) and R972P (nano-sized hydrophobic fumed silica) were selected as the dry coating agents. These silicas were donated by Evonik Corporation (Piscataway, NJ, USA).

### Method

### Milling of Ibuprofen to the Target Size

As particle size is reduced, the interparticle cohesion force increases significantly (Nase et al. 2001). Hence, as the particle size of target API is reduced, the propensity for agglomeration increases significantly. As the previously published studies prove (Yang et al. 2005; Chen et al. 2008; Han et al. 2011) solventless dry coating of the cohesive micronized APIs significantly reduced interparticle cohesion force, resulting in reduced agglomeration. And the differences between coated and uncoated API bulk powder properties are significant when the uncoated APIs size is below 50 μm. Therefore, the particle size of the as-received ibuprofen 50 was milled to ˜10 μm, the particle size which was investigated in the earlier study (Kim et al. 2021).

Dry coating via LabRAM and dry coating formulation

For effective dry coating, a laboratory-scale high energy vibratory mixer, Resodyn acoustic mixer (LabRAM, Resdoyn Corporation, USA) was employed. In a plastic jar, the predetermined amount of API powders and coating materials which were a mixture of A200 and R972P powders were added. The amount of each powder required was calculated by estimating the amount of coating material required to cover the desired percentage of the surface area of the API particles (Yang et al. 2005); (Chen et al. 2008). Then the jar containing those powder mixtures was processed for 5 minutes at the intensity of 75 times the gravitational force, the setting which was determined based on previous work (Capece et al. 2014); (Huang et al. 2015); (Huang et al. 2017).

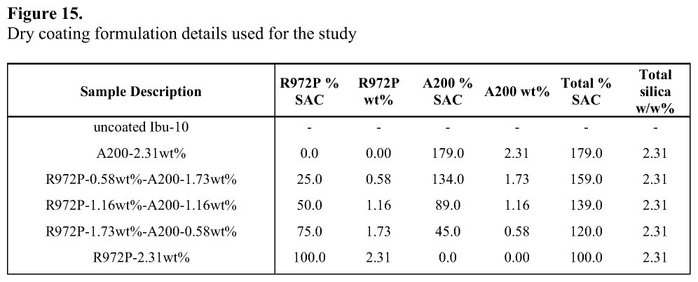

As the first step, calculated the required weight percent of either R972P or A200 to cover 100% of the theoretically available surface area of the API powders (Chen et al. 2008). Then, selecting the higher concentration value, performed dry coating on Ibu10 while varying the weight fraction ratio between hydrophobic and hydrophilic silica. FIG. 15 shows the formulation details. The generated dry coating formulation allowed complete coverage of the API surface, which will allow the study on the impact of the silica mixture formulation without the influence of dry coating quality or efficiency. Once testing the feasibility of inducing a synergistic effect by employing a silica mixture of varying hydrophilicity, repeated the similar test at an optimal surface coating level (Chen et al. 2008); (Han, Ghoroi, and Dave 2013); Kim et al. 2021. or this study, surface area coverage (SAC) was fixed while varying the SAC % ratio between hydrophilic and hydrophobic silica. Details of dry coating formulations are listed in Table 1.

Surface morphology analysis: Field Emission Scanning Electronic Microscope (FESEM)

Before and after drying coated ibuprofen, particle's morphology was studied by employing a Field Emission Scanning Electron Microscope (FESEM) (JSEM-7900f, JEOL Ltd., USA). Before imaging, the sample particles were carbon-coated to enhance imaging quality (Huang et al. 2017). With FESEM, the quality of dry coating can be qualitatively studied as the surface of dry coated and the dispersibility of the groups of particles are visualized. Based on the images gathered by FESEM, the quality of dry coating for each formulation and physical differences between the formulation after dry coating are discussed in the current work.

Particle size distribution analysis: Primary and agglomerated size evaluation

After performing pressure titration upon employing Sympatec Helos/RODOS (Sympatec Inc., NJ), a laser diffraction particle size analyzer that disperses powders with dry compressed air, the primary particle size distributions on the prepared samples were measured. The pressure titration method was reported in the previous work (Huang et al. 2017). The dispersion pressure found through the pressure titration was 1.0 bar, which resulted in the most consistent particle size distribution measurement without causing attrition but successfully dispersing particles to their true size.

For the agglomerated particle size evaluation, Sympatec QICPIC/GRADIS, a dynamic imaging particle sizer (Sympatec Inc., NJ) was employed (Kim et al. 2021). Unlike other available particle sizers that disperse particles with either dry compressed air or liquid medium, QICPIC/GRADIS relies on gravity to disperse particles as they fall through the detection window.

FT4 powder tester: Flowability and bulk density measurements

To evaluate the bulk density and flowability index or flow function coefficient (FFC), which is an indication of how particles are flowing, Freeman FT4 powder tester (Freeman Technologies Ltd., Worcestershire, UK) was utilized. The details of the operation and procedures in the bulk density and FFC measurements are stated in the previous publication (Freeman 2007). FFC is the force ratio between the major principal stress (MPS) and the unconfined yield stress (UYS). In the current study, the pre-consolidation pressure applied for the shear stress testing to MPS and UYS was 3 kPa (Chen, Ding, He, Huang, et al. 2018). The found FFC, by taking the ratio between measured MPS and UYS through FT4 powder tester, serves as the powder flowability classification value, which was defined by Schulze as the following: FFC<1-not flowing, 1<FFC<2-very cohesive, 2<FFC<4-cohesive, 4<FFC<10-easy flowing, and FFC>10-free-flowing (Schulze, Schwedes, and Carson 2008).

Surface contact angle evaluation: Liquid penetration method

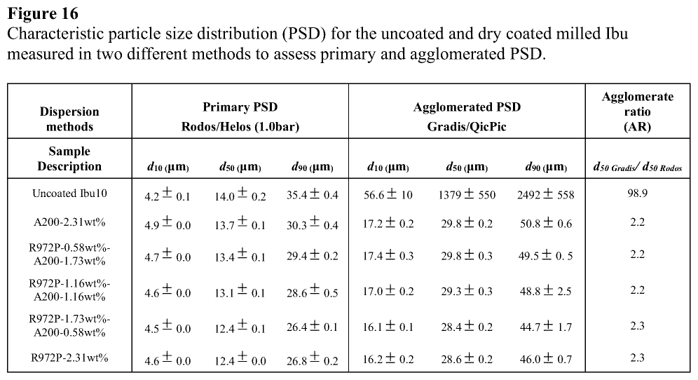

Analysis on a liquid penetration rate as the testing liquid penetrates through a powder bed of interest, shed light on the wettability of the tested powder as per Washburn method explains (Washburn 1921). For the wettability assessment, Attension Sigma 700 (Biolin Scientific, Linthicum, MD, USA) was utilized and followed the experimental method that was previously established in the earlier work, Kim et al. 2021. In the current study, a reference liquid, saturated n-hexane with ibuprofen was used to completely wet the testing powders which allows calculation of packing factor. Once the packing factors at the corresponding powder samples were found, the liquid penetration test was performed with the testing liquid, the saturated de-ionized water with ibuprofen. During the test, the testing liquid was kept constant while the testing powders were varying. The properties of the employed liquids are shown in FIG. 16.

Ture density measurements: porosity evaluation

The FT4 testing can assess the bulk powder flowability and the conditioned bulk powder density (Pbulk density). A bulk powder density is a ratio between the mass of the powder bed to the volume of the powder bed. The volume of the powder bed includes the volume of powder volume and the volume of voids.

$$Porosity = 1 - ( \frac{{ρ}_{bulk density}}{{ρ}_{True density}} ) = 1 - \frac{{V}_{true particles}}{{V}_{true particles} + {V}_{voids}} \qquad ( 1 )$$

Equation 1 shows the porosity of the powder beds can be calculated by employing the experimental bulk and true powder densities. It is reported that the geometric packing factor of powder beds is proportional to the bed's porosity, impacting the surface contact angle (Kirdponpattara, Phisalaphong, and Newby 2013). In this study, true powder density was measured to evaluate the powder porosity to study the relationship between porosity and contact angle at varying dry coating formulation. Thus, operated a Multipycnometer (P/N 02029-1, Quantachrome Instrument, USA) in the helium environment and measured true powder densities. Five repeated measurements were taken to ensure repeatability, and the averaged values were reported.

### Dissolution Study

To maximize discernment between the formulations, de-ionized water was used as the dissolution medium given poor solubility of Ibu in water. For the dissolution, USP IV flow-through-cell method was employed, and the testing condition strictly followed USP<700>guideline. 1 L of de-ionized water was prepared as the medium, and 15 mg of Ibu powders were added in each cell, allowing the medium to flow through the cell and dissolve the powder over the period of 900 minutes.

During the entire dissolution process, the temperature kept at 37.0±0.1° C. and the flow rate of the medium was maintained at 16 mL/min. The details of the experimental setup are stated in the earlier work, Kim et al. 2021.

### Results

### Hypothesis 1

Under the premise that coating effectiveness-reduction in the interparticle cohesion force is significant—is guaranteed, with silica mixture dry coating, predicted the enhancement in flowability index (FFC) and bulk density (BD) is proportional to the weight % of hydrophobic silica while the API release rate is proportional to the weight % of hydrophilic silica. Therefore, the greatest FFC and BD were expected when only hydrophobic silica covered the surface of Ibu10. Likewise, the fastest dissolution rate was assumed when Ibu10 particles were covered by hydrophilic silica only. Ensuring sufficient coverage of the API particle's surface by adding 2.31% by weight of silica (see FIG. 15) eliminated the issue concerning insufficient coating.

Surface morphology after dry coating: Fixed silica weight % cases

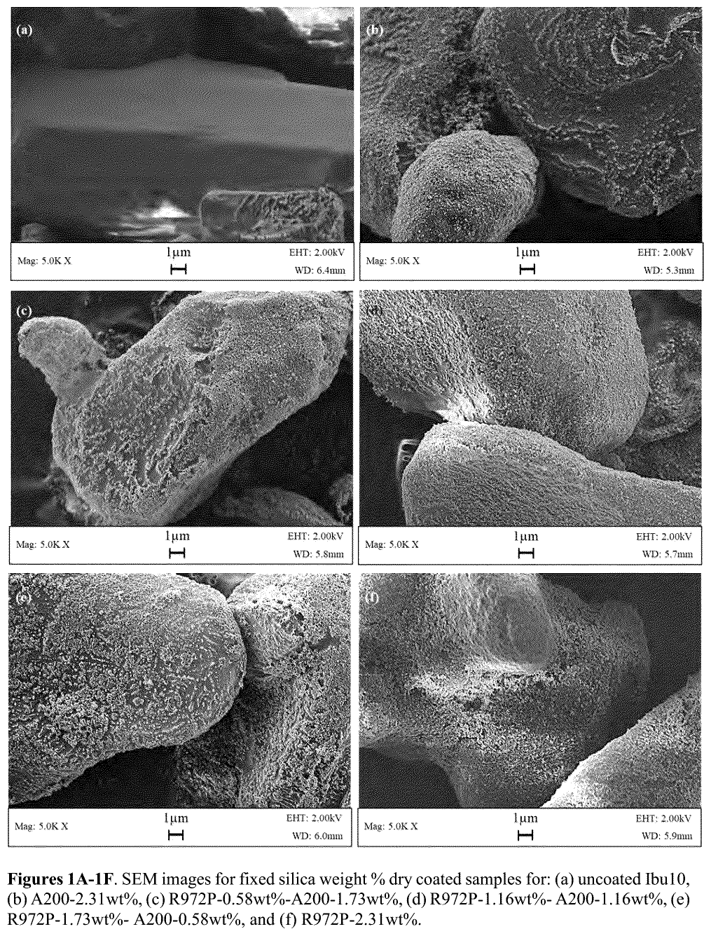

The surface morphology of dry coated Ibu10 at each formulation was analyzed under SEM. FIG. 1 presents the surface image of each sample. As intended, all surface images of the dry coated Ibu10 appeared to be sufficiently covered by silica. Under SEM analysis, no observable differences were found when silica mixture compositions were varied (see FIG. 1). Thus, it was concluded that any differences in agglomerate size, FFC, BD, surface contact angle, and dissolution profile between the samples resulted from the hydrophilicity of the silica mixture, not due to the dry coating quality or surface morphology difference.

Primary and agglomerated size evaluation: Fixed silica weight % cases

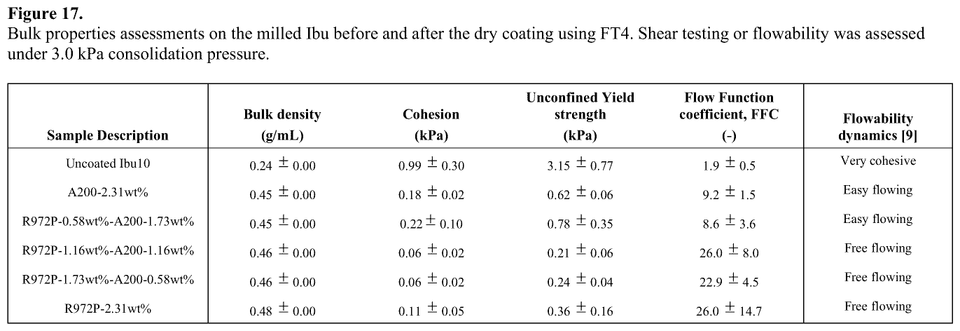

Employing both compressed air dispersion and gravity-driven dispersion methods, primary and agglomerate particle size distributions were evaluated, respectively. FIG. 17 summarizes the resulted measurements from two dispersion methods. As the results from the compared air dispersion method show, the dry coating did not alter the inherent particle size distribution of Ibu10 for all formulations. But in the case of agglomerated particle size distribution, which was evaluated via the gravity dispersion method, a significant reduction in the size measurements and the randomness was observed for all dry coated samples. This confirmed that irrespective of silica mixture composition, the dry coating reduced the interparticle cohesion force effectively (Yang et al. 2005); (Chen et al. 2008).

### FT4 Results: Fixed Silica Weight % Cases

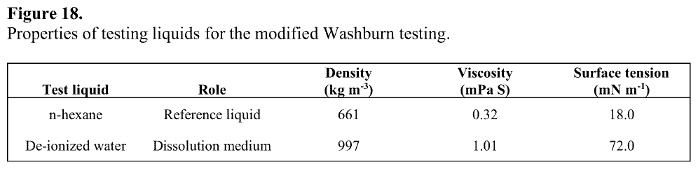

Employing a powder rheometer (FT4) powder bulk density, and flowability were assessed. Before measurements, the powders were conditioned by breaking up the previously formed particle contacts, ensuring repeatable evaluations on the bulk density measurements and shear testing. FIG. 18 presents the summarized FT4 analysis results on fixed silica weight % cases. As it was evident in the agglomerated particle size evaluation results shown in FIG. 17, analysis done by FT4 also demonstrates significant reduction in the interparticle cohesion force. For instance, the unconfined yield strength, which was evaluated at the pre-consolidation pressure of 3 kPa, was reduced by the order of magnitude. Likewise, the cohesion calculated based on the shear stress analysis from generating a tangent line to the Mohr's curves showed significant reduction after dry coating (Freeman 2007). In addition, notable increase in bulk density results after dry coating suggests significant reduction in the void volume, inferring that the easy of compaction of the powder samples had increased after dry coating (Chen, Ding, He, Huang, et al. 2018).

### Surface Contact Angle Measurement: Fixed Silica Weight % Cases

The dissolution process of a material is the result of the joined phenomena including surface wetting, liquid penetration, diffusion through the solution, and so on (Forny, Marabi, and Palzer 2011); (Lazghab et al. 2005). Especially, at the incipient of dissolution process, two phenomena are the critical contributors, which are the surface wetting of the material and the solvent penetration through pores of the material (Lazghab et al. 2005); (Siebold et al. 1997). And these two phenomena can provide information on surface hydrophilicity or affinity of the solvent, and the presence of agglomerates, respectively (Thakker et al. 2013); (Ji et al. 2016); (Siebold et al. 1997). In this study, focusing on the assessment on the surface affinity to the water or hydrophilicity at each coating formulation (Thakker et al. 2013); (Li et al. 2015), modified Washburn method was employed.

$${m}^{2} = ( \frac{C {ρ}^{2} γcosθ}{η} ) t \qquad ( 2 )$$

In equation 2, n, p, and y are, respectively, the viscosity, the density, and the surface tension of the wetting liquid; C is a geometric packing factor; 0 is the surface contact angle in radian; m is the total mass of the wetting liquid that penetrates through the packed bed. In the current study, testing liquid kept constant while powder samples were varied. Thus, all the liquid terms were considered to be constant while the geometric packing factor, C, was varying with dry coating formulations.

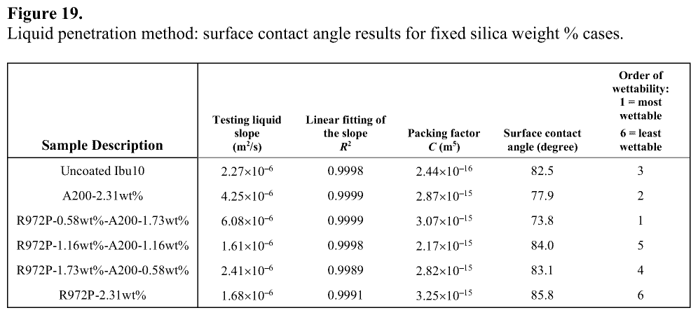

Therefore, before the surface contact angle assessment, C was experimentally found by employing the saturated n-hexane, which completely wets the surface of the testing powder resulting in cose term to be 1 (Amaro-Gonzalez and Biscans 2002). Then, assuming C stays constant for each dry coated sample, found the surface contact angles by employing the saturated de-ionized water. FIG. 19 shows the summarized results. The surface contact angle resulted revealed an unexpected outcome.

Referring to the samples by ID number, Sample 1-1 was dry coated with only hydrophilic silica while Sample 1-2 was dry coated with the mixture silica which contains hydrophilic and hydrophobic silica by 1 to 3 weight percent. Intuitively, one might expect to see the lowest surface contact angle or highest surface hydrophilicity with Sample 1-1 as it has the greatest concentration of hydrophilic silica on the surface of API. On the contrary, the experiment showed the lowest contact angle with Sample 1-2, which has ⅓ of hydrophobic silica within the silica mixture used for dry coating. In conjunction with the agglomerate PSD results which are shown in FIG. 17 it is evident that the agglomerate size is smaller in Sample 1-2 compared to that of Sample 1-1. As it is reported, surface wettability of powders is a complex phenomenon which are affected by particle shape, surface roughness, porosity, and particle size (Chau et al. 2009); (Kirdponpattara, Phisalaphong, and Newby 2013) apart from polarity. Assuming both the surface the surface roughness and shape are comparable for Samples 1-1 and 1-2, difference in their agglomerate particles could have caused the difference in the number of capillary pores or porosity in which the liquid penetrates (Kirdponpattara, Phisalaphong, and Newby 2013); (Forny, Marabi, and Palzer 2011). As the result, despite the greater number of available hydrophilic silica on the surface of API in Sample 1-1, because of smaller agglomerate size in Sample 1-2, it is more likely to have greater number of capillary pores within Sample 1-2 compared to Sample 1-1, which explains faster penetration rate in Sample 1-2. This phenomenon further validated by the dissolution rate evaluation which is discussed in the following section.

### Dissolution Behavior Evaluation: Fixed Silica Weight Cases

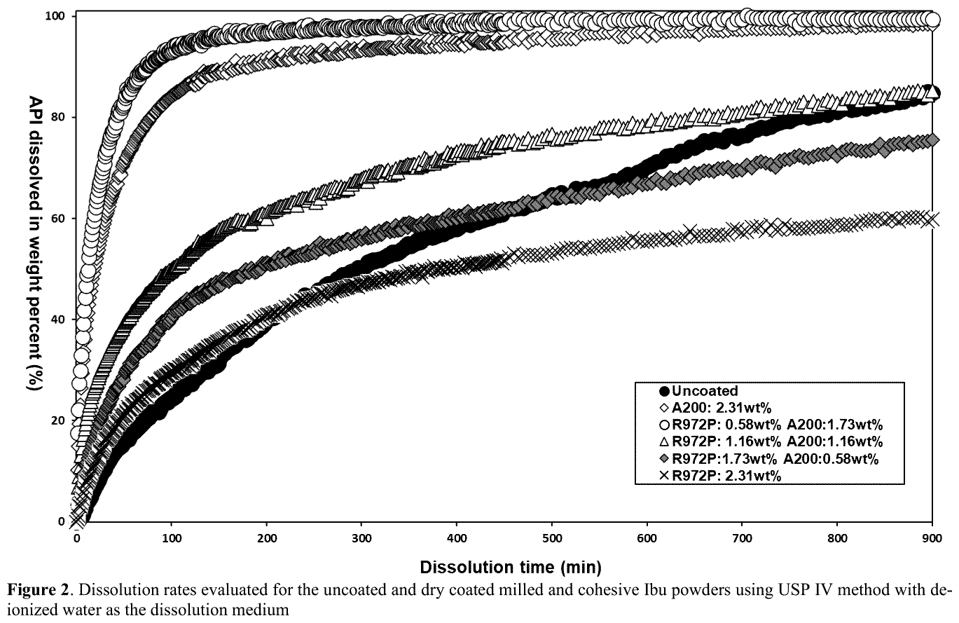

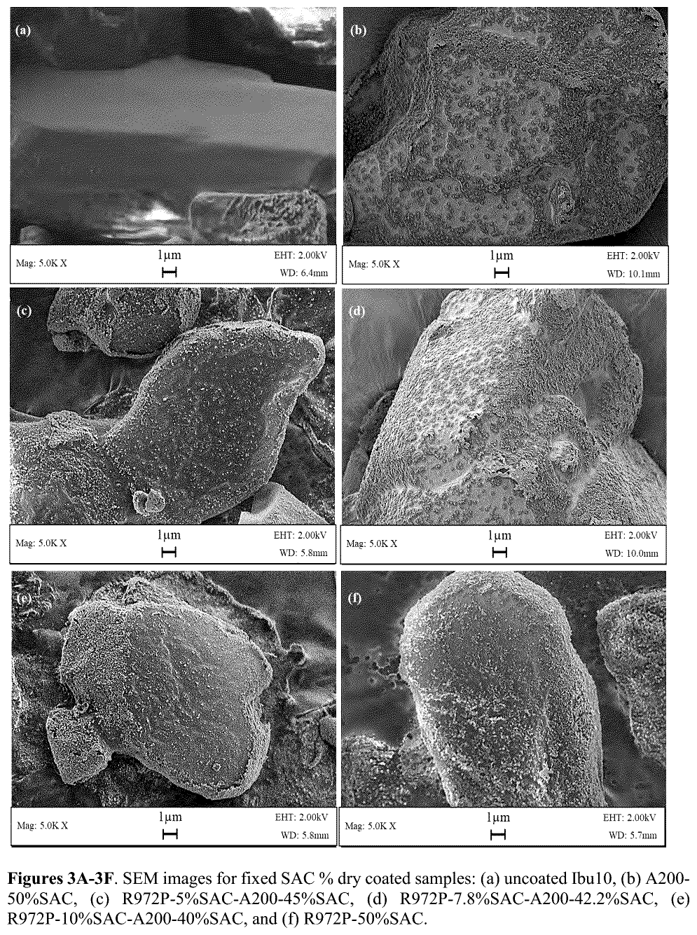

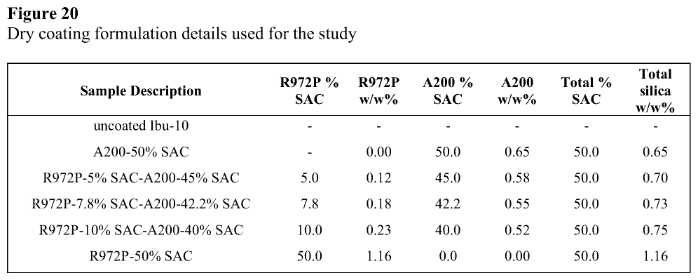

Employing the USP IV method, studied dissolution profiles of the dry coated Ibu10 at silica weight %. FIG. 2 presents the resulted dissolution profiles for all cases. Contrary to the intuitive prediction, the silica mixture case, Sample 1-2, showed the fastest dissolution rate followed by only hydrophilic dry coating, Sample 1-1. FIG. 20 shows calculated the area under the curve (AUC), which clearly illustrated the faster dissolution for the mixed silica dry coating case (Sample 1-2) compared to the hydrophilic silica dry coating only (Sample 1-1). The additional finding observed during the dissolution process is the initial API dissolution rate. In all cases, for the first 250 minutes into the dissolution, the dissolution rates of dry coated Ibu10 were faster than that of uncoated Ibu10. Kim et al. 2021 also reported a similar phenomenon, which was explained by the decrease in agglomerate size after the dry coating with the measurement data. After 250 minutes, dissolution of all the loose fines completed, the dissolution of the samples containing higher concentrations of hydrophobic silica started to slow down as the increase in their surface hydrophobicity outweighed the impact of agglomeration reduction. Sample 1-2 showed the fastest initial API release amongst the coated group, demonstrating the combination of hydrophilic and hydrophobic silica can induce a synergistic effect. Hydrophobic silica reduces the agglomerate size while hydrophilic silica improves the surface wettability, thus expediting the dissolution process.

### Hypothesis 2

Accompanied by agglomerate size evaluation as well as the surface contact angle and dissolution behavior, the first part of the current study demonstrated the feasibility of achieving a synergistic effect from a silica mixture of varying hydrophobicity to expediated the dissolution rate even further. As FIG. 1 shows, the surface of API seemed to be completely covered by silica and on some regions, the silica agglomerates are evident. For instance, some of the previous studies showed that having too much silica not only causes them to agglomerate (Deng, Zheng, and Dave 2018) but processing them with a high-intensity mixer such as LabRAM acerbate the silica agglomeration (Zheng et al. 2020). Besides, although nano-sized fumed silica was reported to because no harmful side effects on the biological system, it is wise to control the level of silica concentration included in the daily dosage of pharmaceutical products to avoid any possible health hazardous impacts (Chen, Liu, et al. 2018). What is more, both previously reported theoretical (Chen et al. 2008) and experimental (Kim et al. 2021) showed that beyond a certain level of surface area coating, there is no notable bulk powder properties improvement.

Therefore, in the latter part of the current study, the theoretical SAC& was adjusted to 50% and set as constant based on the mentioned previous studies. Then, the SAC % ratio between hydrophobic and hydrophilic silica was varied following the observed trend from the former part of the current investigation. Table 1 summarizes the detailed dry coating formulation.

Surface Morphology after Dry Coating: Fixed SAC % Cases

FIGS. 3A-3F show the SEM images on the dry coated samples at fixed SAC % with the hydrophobic and hydrophilic silica components variations. Compared to FIG. 1, even and discrete silica coating is evident, and agglomeration of silica seemed to be smaller compared to that of the previous case study. The SEM images (see FIGS. 3A-3F) provided a qualitative reference to conclude that the intended dry coating level was achieved.

### Primary and Agglomerated Size Evaluation: Fixed SAC % Cases

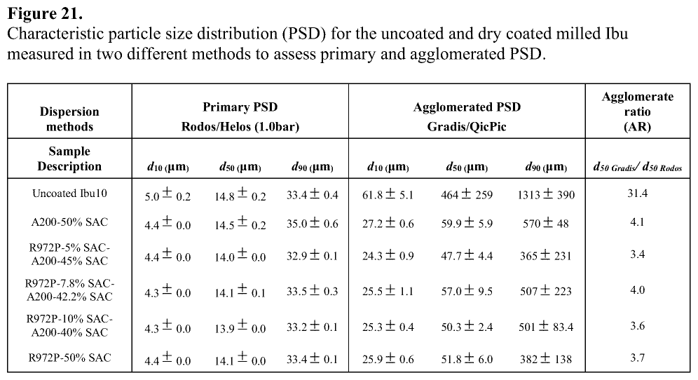

Both primary and agglomerated particle sizes were evaluated to observe if the selected SAC& was enough to reduce the interparticle force between the particles effectively thus significantly reducing agglomerate size. FIG. 21 summarizes the particle size measurement results for each case. Compared to the measurement data on the fixed silica weight & cases from the former part of the investigation, the measured agglomerated particle sizes were larger while the primary particle sizes were within a similar range. But in both cases, the agglomerate particle sizes were reduced by one or more order of magnitude. This shows that 50% SAC coverage cases reduced the surface energy significantly, effectively breaking agglomerate sizes.

### FT4 Results: Fixed SAC % Cases

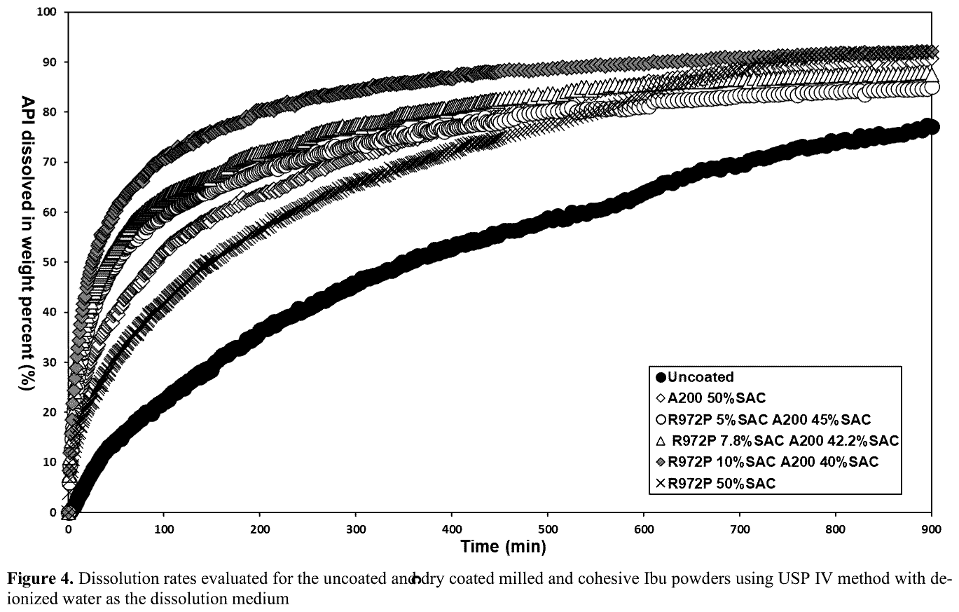

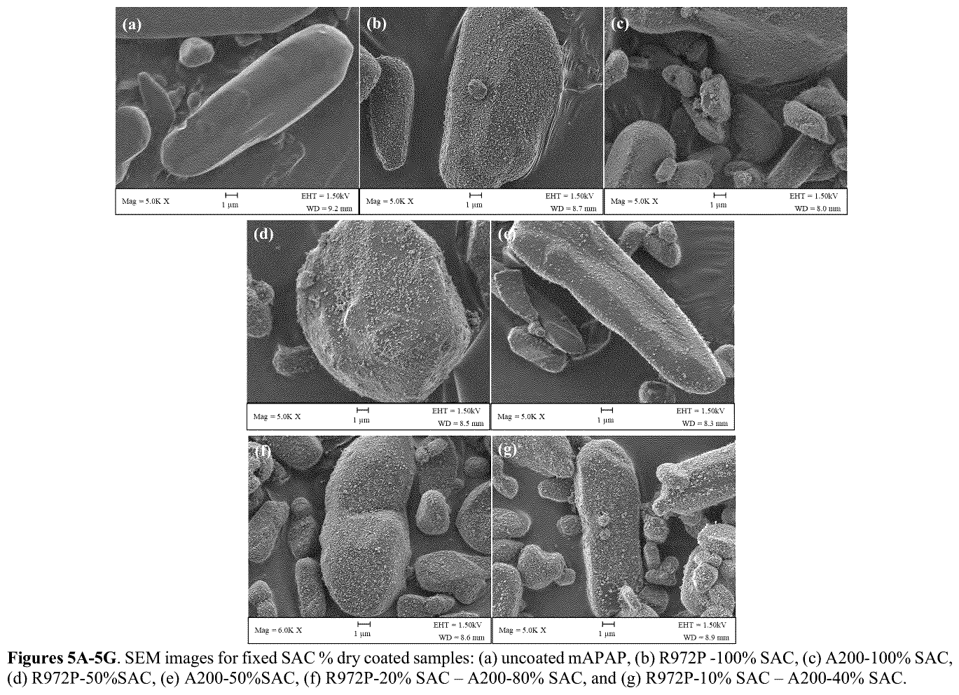

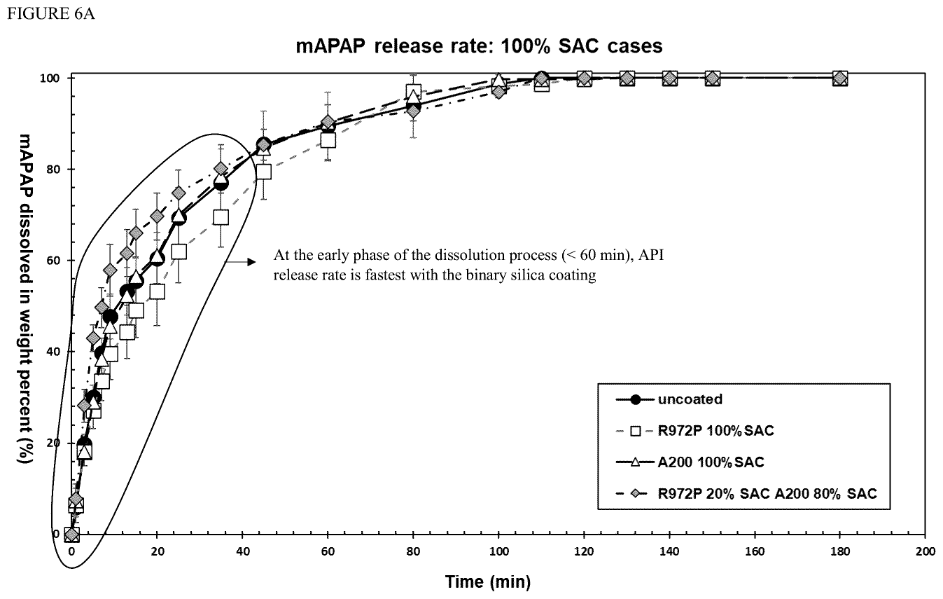

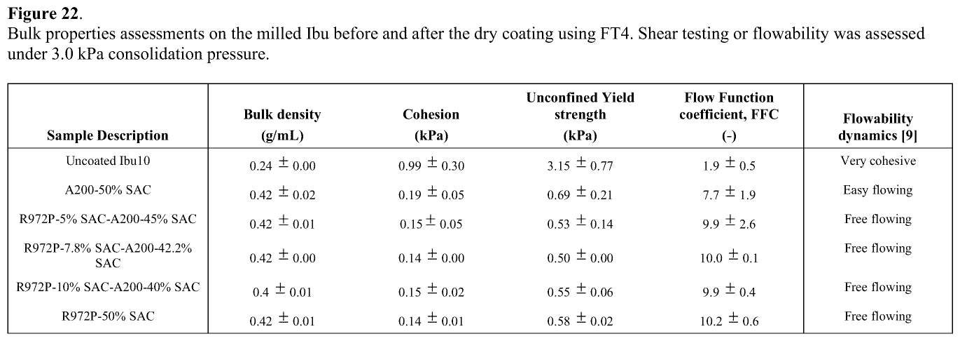

As the agglomerate evaluations indicated, measured bulk density and flowability from further FT4 validated the effectiveness of the dry coating at SAC % for all samples. The results are shown in FIG. 22. To compare the improvement in powder flowability (FFC) between the cases, generated FIG. 4. In FIG. 4, both normalized FFC with respect to the uncoated Ibu10 and actual value of FFC are shown. For all dry coated API, irrespective of the dry coating formulation, the improvement in flowability is evident as the flow regime changed from cohesive to well flowing or free-flowing region. In the case of fixed silica weight cases (Samples 1-1 through 1-5), the composition differences seemed to have no significant impact on flowability improvement even when only hydrophilic only and hydrophobic only coated samples, Sample 2-1 and Sample 2-5 were compared, respectively. However, in case of fixed silica weight % cases, the cases where the surface of API was completely covered by the silica, silica composition seemed to have a notable impact on the flowability improvement of the cohesive API powders as some groups showed a shift in the flow regime from cohesive to free-flowing while the rest showed a shift from cohesive to well flowing. It seemed that for Samples 1-1 through 1-5 because the surface of API was completely covered by silica, silica properties seemed to govern the flow behavior of the API after dry coating as the fluctuating flowability with the silica composition conveys. However, fixed SAC % coated Samples 2-1 through 2-5, the flowability does not depend on the silica composition, but rather, they were all within the comparable ranges, belonging same flowability regime. This comparison between the two cases revealed a remarkably interesting finding regarding the powder blend formulation. Typically, the concentrations of excipients like silica are decided based on the toxicological evaluations (Chen, Liu, et al. 2018) under the approval and regulations of a government agency. And this guideline is given as the weight percent. The flowability comparison between two cases from the current study showed that in addition to the toxicological concern, it is important to find the optimal SAC % when the dry coating method is applied so that the flowability of the bulk powder can be easily predicted and controlled, insensitive of the silica composition used.

### Surface Contact Angle Measurement: Fixed SAC % Cases

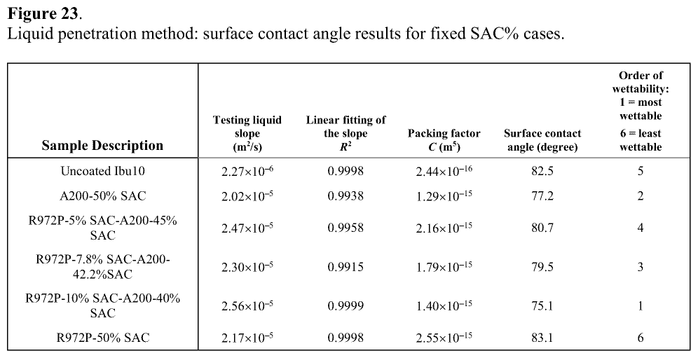

Employing the modified Washburn method, geometric packing factors and surface contact angles were assessed for Samples 2-1 through 2-5. FIG. 23 shows the summarized results from the experiment. The pattern observed regarding the evaluated contact angle for fixed SAC % cases confirms that was found with the former study when silica weight % was fixed. The surface contact angle was for Sample 2-4, which had a silica mixture, with a SAC % ratio of 1 to 4, R972P to A200, respectively. This phenomenon could be explained by the measured agglomerate size, shown in FIG. 21. Referring to FIG. 21, Sample 2-4 shows the smallest agglomerate size, which could have benefitted liquid penetration rate through capillary porous of the powder pack bed (Forny, Marabi, and Palzer 2011). Further evaluation continued to see how the porosity affected due to dry coating formulations.

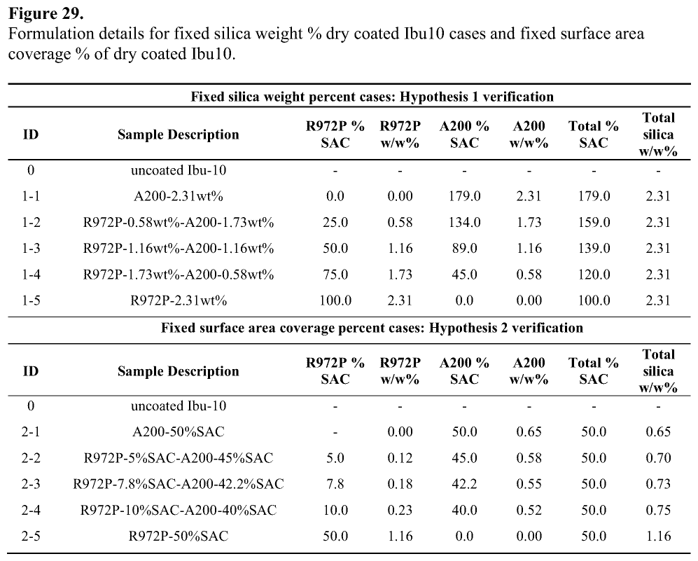

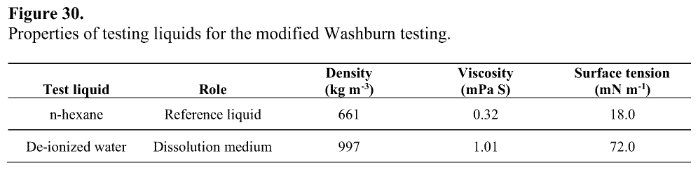

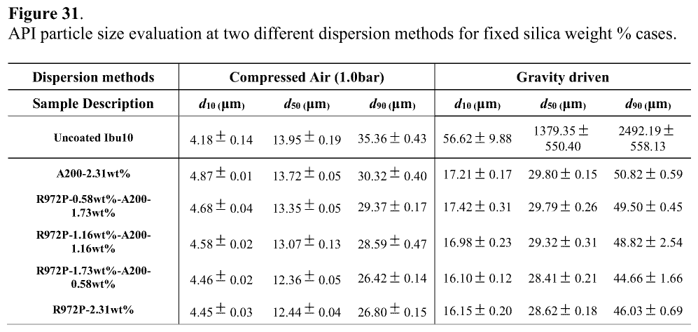

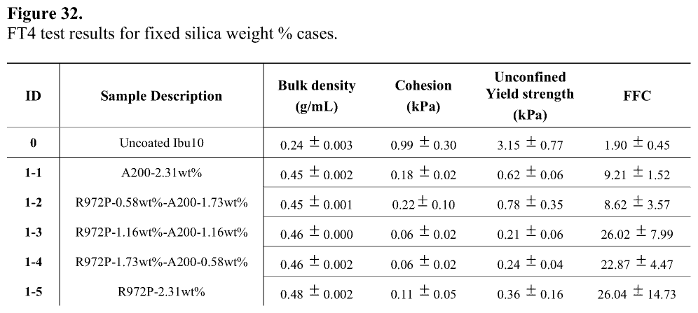

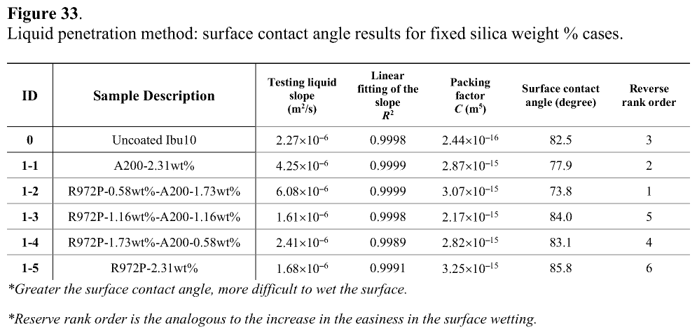

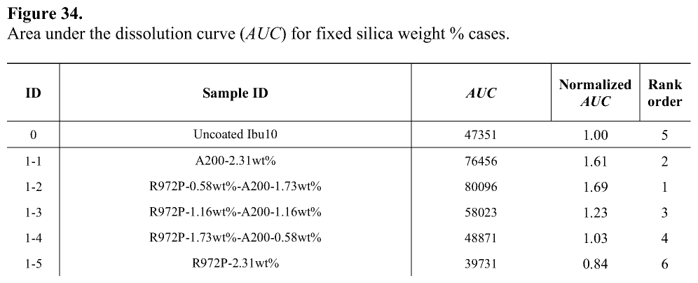

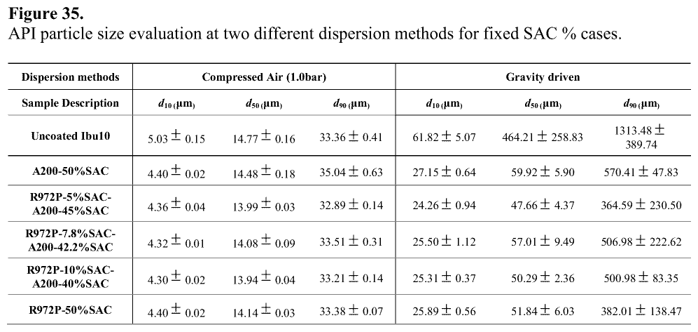

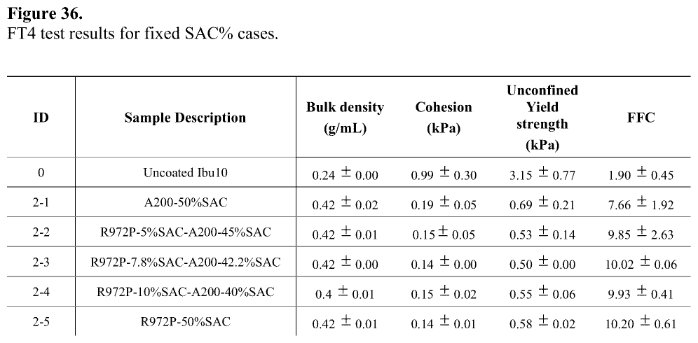

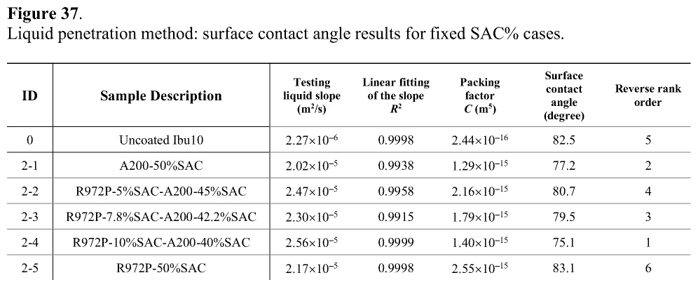

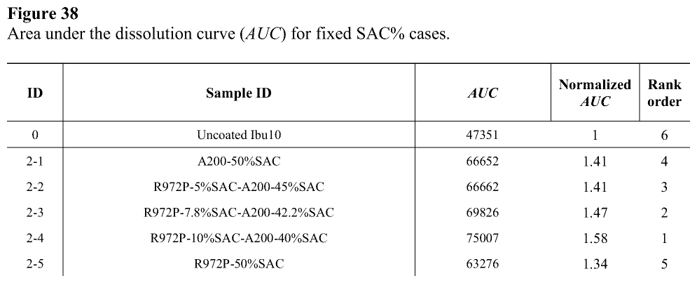

FIG. 29 illustrates a table showing formulation details for fixed silica weight % dry coated Ibu10 cases and fixed surface area coverage % of dry coated Ibu10. FIG. 30 illustrates a table showing properties of testing liquids for the modified Washburn testing. FIG. 31 illustrates a table showing API particle size evaluation at two different dispersion methods for fixed silica weight % cases. FIG. 32 illustrates a table showing FT4 test results for fixed silica weight % cases. FIG. 33 illustrates a table showing Liquid penetration method: surface contact angle results for fixed silica weight % cases. FIG. 34 illustrates a table showing Area under the dissolution curve (AUC) for fixed silica weight % cases. FIG. 35 illustrates a table showing API particle size evaluation at two different dispersion methods for fixed SAC % cases. FIG. 36 illustrates a table showing FT4 test results for fixed SAC % cases. FIG. 37 illustrates a table showing Liquid penetration method: surface contact angle results for fixed SAC % cases. FIG. 38 illustrates a table showing Area under the dissolution curve (AUC) for fixed SAC % cases.

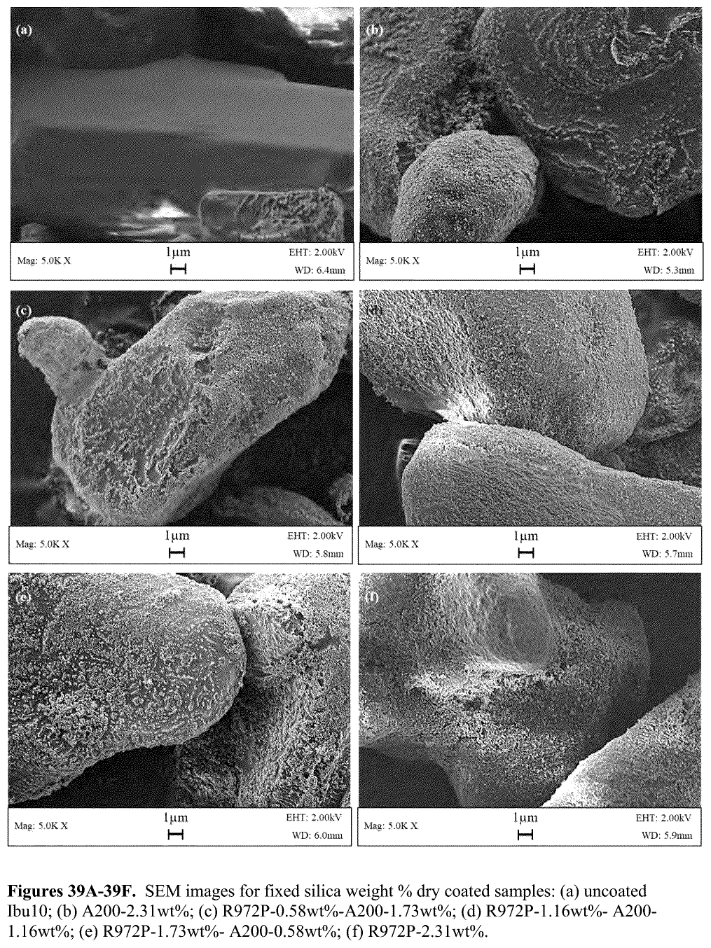

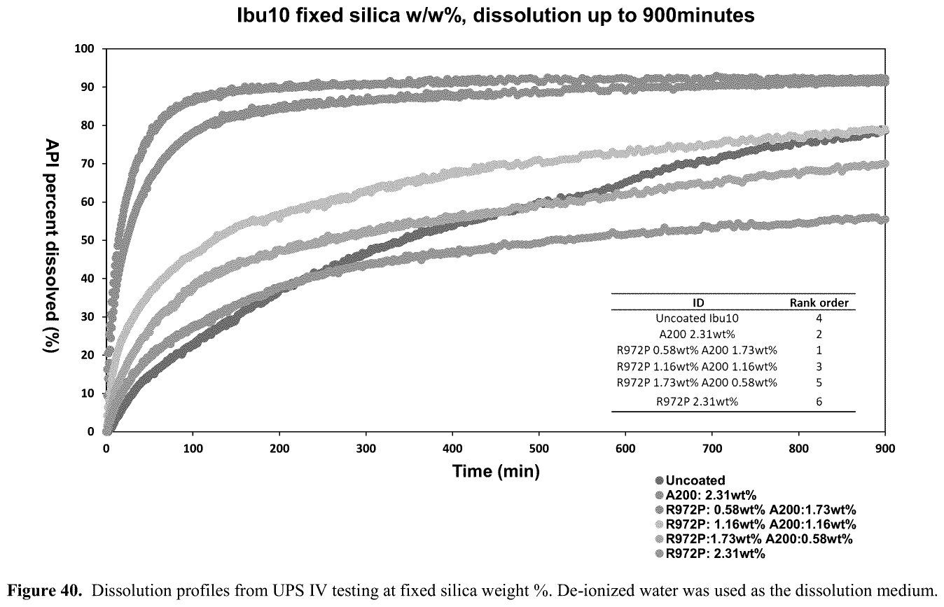

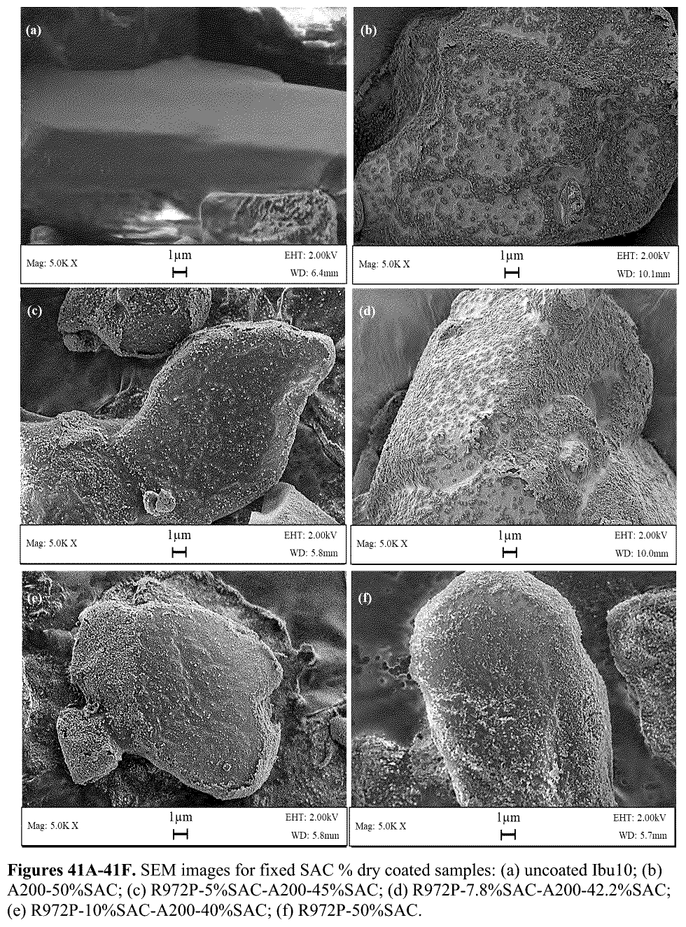

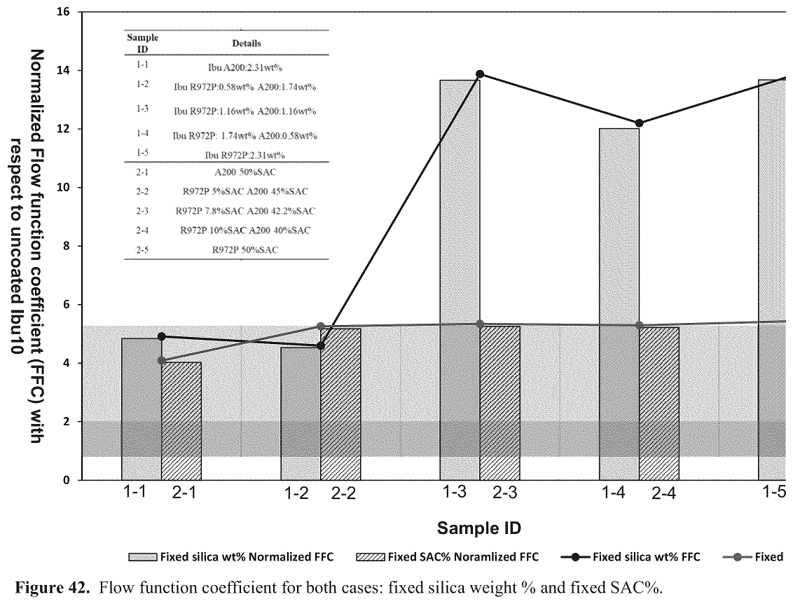

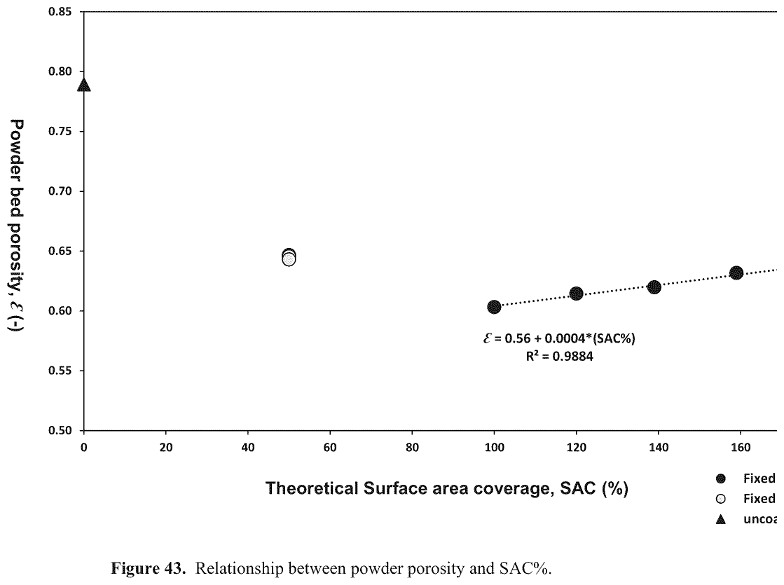

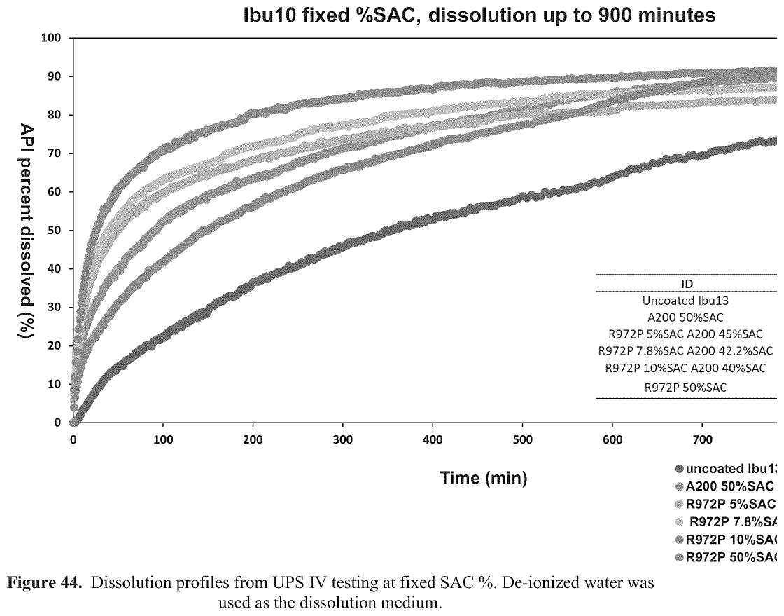

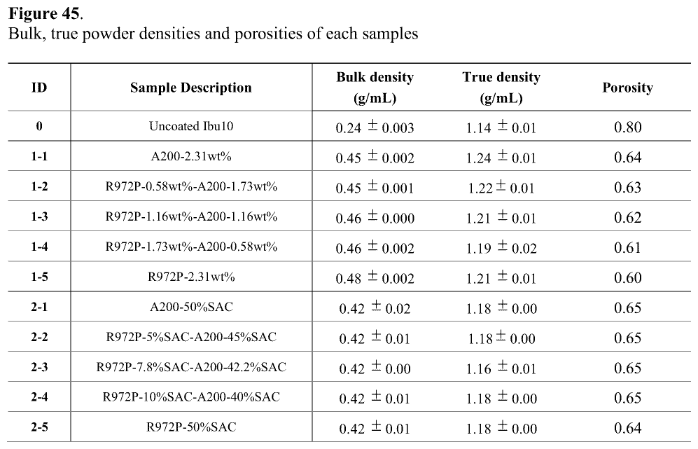

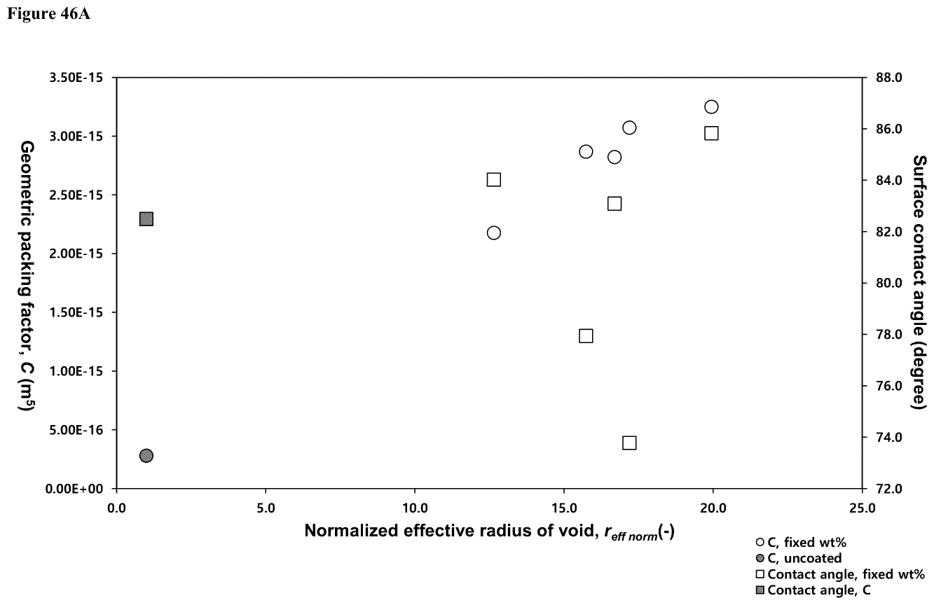

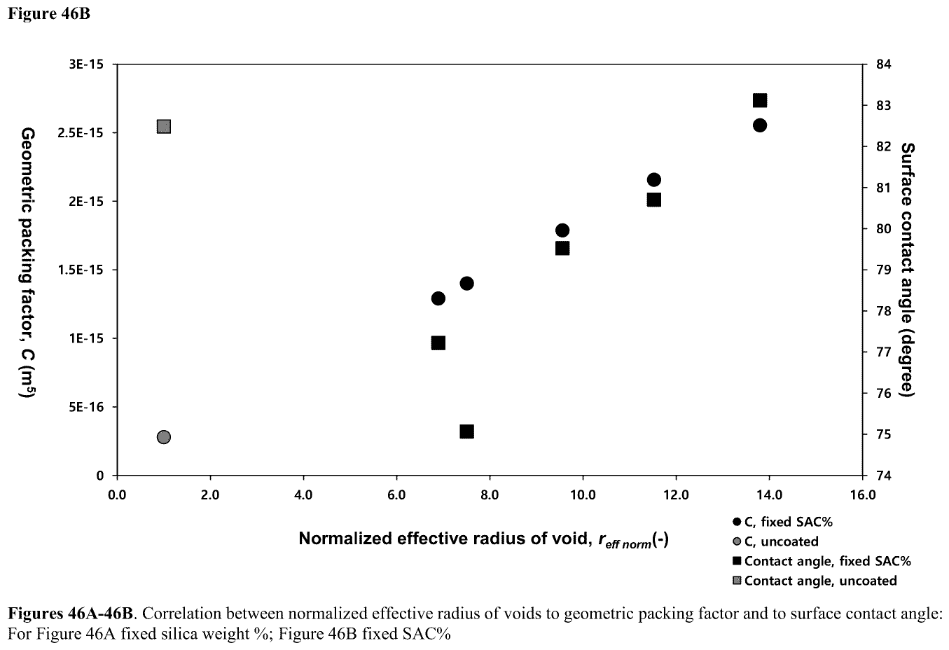

FIGS. 39A-39F illustrates SEM images for fixed silica weight % dry coated samples, respectively: (a) uncoated Ibu10; (b) A200-2.31 wt %; (c) R972P-0.58 wt %-A200-1.73 wt %; (d) R972P-1. 16 wt %-A200-1.16 wt %; (e) R972P-1.73 wt %-A200-0.58 wt %; (f) R972P-2.31 wt %. FIG. 40 illustrates dissolution profiles from UPS IV testing at fixed silica weight %. De-ionized water was used as the dissolution medium. FIGS. 41A-41F illustrate SEM images for fixed SAC % dry coated samples, respectively: (a) uncoated Ibu10; (b) A200-50% SAC; (c) R972P-5% SAC-A200-45% SAC; (d) R972P-7.8% SAC-A200-42.2% SAC; (e) R972P-10% SAC-A200-40% SAC; (f) R972P-50% SAC. FIG. 42 illustrates Flow function coefficient for both cases: fixed silica weight % and fixed SAC %. FIG. 43 illustrates a relationship between powder porosity and SAC %. FIG. 44 illustrates dissolution profiles from UPS IV testing at fixed SAC %. De-ionized water was used as the dissolution medium. FIG. 45 illustrates a table showing bulk, true powder densities and porosities of each sample listed. FIGS. 46A-46B illustrates plot graphs showing the correlation between normalized effective radius of voids to geometric packing factor and to surface contact angle, respectively, for FIG. 46A fixed silica weight %; and FIG. 46B fixed SAC %.

### Density Based Porosity Evaluation

The porosities of the powder beds before and after dry coating were preliminarily calculated based on their density values (see Supplementary materials section, FIG. 45). Then, the relationship between porosity and SAC % was analyzed as shown in FIGS. 5A-5F (for SAC %, see FIG. 15). Referring to FIGS. 5A-5F, it is evident that dry coating significantly reduced the porosity, which will aid the compaction process for tablet production. In FIGS. 5A-5F, it is interesting to note that for fixed SAC % cases, the porosities were almost unvarying. However, for the fixed silica weight % case, porosities seemed to increase linearly with increasing SAC8. More studies with varying API sizes should be conducted further, to validate the observed trend. However, based on the current results shown in FIGS. 5A-5F, it seemed to show the possibility of modulating the porosity of the powder bed through SAC& selection, irrespective of the type of the nano-sized coating materials. This is a meaningful finding since the porosity is not only related to the liquid penetration rate (Washburn 1921); (Forny, Marabi, and Palzer 2011); (Kirdponpattara, Phisalaphong, and Newby 2013); but also to the compatibility (Chen, Ding, He, Huang, et al. 2018); (Chen, Ding, He, Fan, et al. 2018) of the powder.

Further analysis is based on the modified Washburn method.

$$C = \frac{{r}_{eff} {A}^{2} {ε}^{2}}{2} \qquad ( 3 )$$

### (Kirdponpattara, Phisalaphong, and Newby 2013)

Where C is the geometric packing factor, reff is the effective radius of voids, A is the surface area of the packed bed and & is the porosity of the packed bed. In the current study, first found reff then found normalized reff by diving the calculated reff A-of the coated by reff A-of the uncoated Ibu10 (see Supplementary Materials, FIGS. 46A-46B)). FIGS. 46A-46B preliminarily convey that the wetting depends on the capillary radius of the pores, which are strongly affected by the dry coating formulation, not necessarily the dry coating material's type. Figure S1(a) and (b) preliminarily convey that the wetting depends on the capillary radius of the pores, which are strongly affected by the dry coating formulation, not necessarily on the dry coating material's type. However, more detailed studies will be required to validate this preliminary observation.

### Dissolution Behavior Evaluation: Fixed SAC % Cases

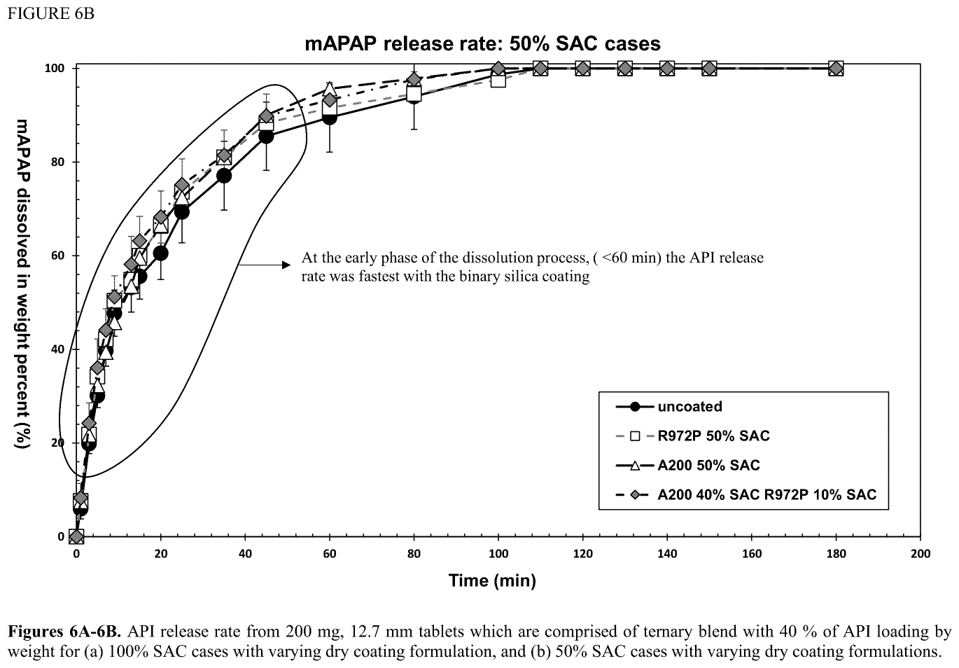

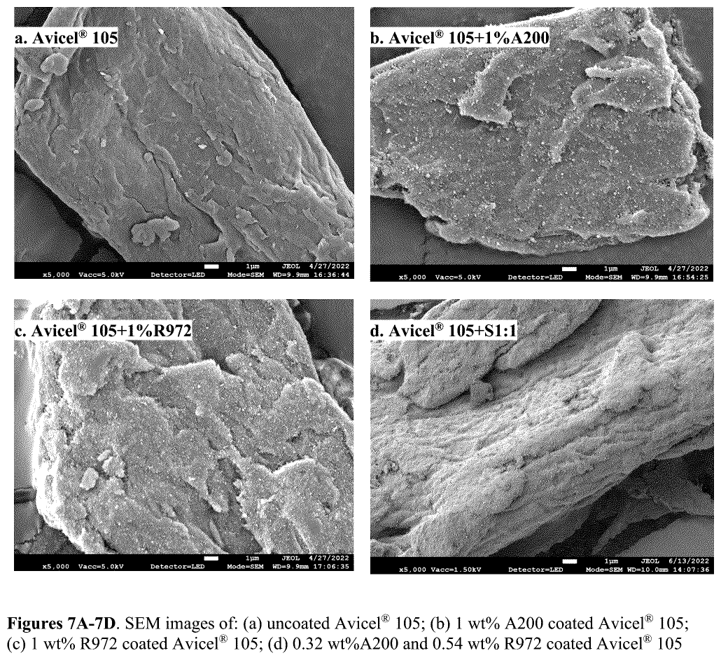

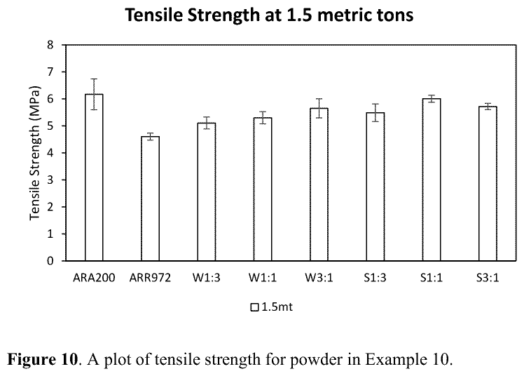

Employing the USP IV method, evaluated the dissolution rates of fixed SAC % cases in de-ionized water. FIGS. 6A-6B show the measured dissolution profiles. FIG. 10 shows the summarized results of the calculated area under the dissolution curves (AUC). Intuitively, one might expect the faster API release rate for the fixed silica weight % dry coated cases when dry coated with only hydrophilic silica (A200). However, the observed dissolution profiles showed that for all the fixed SAC % samples, the API dissolution process was faster than that of the fixed silica weight % groups. Both the SEM images (see FIG. 1) and the previous studies (Deng, Zheng, and Dave 2018); (Zheng et al. 2020) an explain this phenomenon by the existence of the silica agglomerates on the surface of API. The fixed silica weight % cases have higher silica concentration than the fixed SAC % cases (see FIG. 1). Referring to FIG. 1, it seemed that the excess silica particles formed agglomerates with one another on the surface of API. But for fixed SAC % cases (see FIG. 3, FIGS. 7A-7D, FIGS. 11A-11C, and FIG. 39), the API surface was discretely coated, and the likelihood of silica forming agglomerates seemed significantly lower than in the former. Both studies (Deng et al. 2018; Zheng et al. 2020) provide computational and experimental evidence for silica agglomeration when there is a high concentration of silica and dry coated under high energy. Apart from the mentioned SEM images and literature work, an erratic trend between r_{eff} and surface contact angle also infer the presence of irregular silica agglomerates for high silica concentration samples (see FIG. 46A). Overall, comparing fixed silica weight % and fixed SAC % cases, the excess amount of silica, although they successfully reduced interparticle cohesion force as the FFC values indicate (see FIG. 18), could limit the dissolution process improvement by forming silica agglomerates on the surface of API thereby hindering wetting of the API thus affecting the dissolution process.

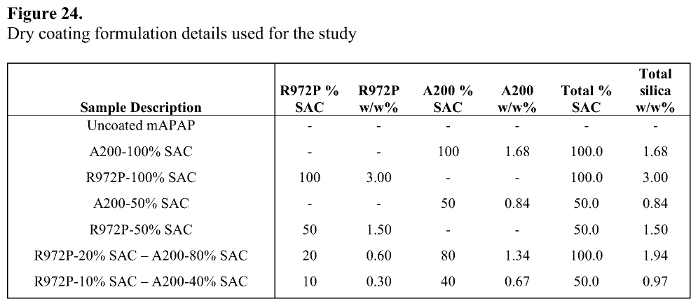

Another intriguing observation seen from FIGS. 6A-6B and FIG. 24 is that all dry coated samples dissolved faster than uncoated API, even with only a hydrophobic silica coating. This phenomenon also was reported elsewhere (Kim et al. 2021); (Qu et al. 2015) that could be explained by the API agglomerate reduction. An additional intriguing find from the dissolution study is that the mixed silica dry coated groups resulted in a faster API release rate than hydrophilic silica-only API. Referring to the agglomerate particle size measurement (see FIG. 21) and surface contact angle evaluations (see FIG. 23), it is evident that when hydrophilic and hydrophobic silica were mixed, they work synergistically by reducing agglomerate size led by hydrophobic silica and improving wetting rate by hydrophilic silica. Also, there is an optimal ratio between hydrophobic to hydrophilic silica ratio, which maximizes the collaborative effect as the difference between Sample 2-2 through Sample 2-4 convey.

Overall, the mechanical properties and dissolution results from the fixed SAC % groups convey that when the surface of the model API (Ibu10) is dry coated about 50% SAC, the interparticle cohesion force is reduced significantly, and formed a small number of silica agglomerates on the surface of API. Consequently, those discretely and sparingly dry coated API showed a faster dissolution rate than compared to that of the model API, which was completely dry coated by silica.

The current study of the present inventors focused on evaluating the feasibility of inducing synergistic effects when the dry coating materials used were hydrophilic and hydrophobic silica mixtures.

The experimental results showed that the powder properties improvements after dry coating with silica mixture were as significant as a single type of silica dry coating. But more interestingly, there were two notable phenomena from the current study.

The first discovery was the faster dissolution rate when silica mixture was used as the dry coating material. Both the fixed silica weight % and fixed SAC % cases convey, it seemed there is a preferable silica composition that can maximize the collaborative impacts when hydrophilic and hydrophobic silica were mixed.

The second discovery was the silica agglomeration effect. Comparing the bulk powder properties results, including FFC, agglomerate size, surface contact angle, and AUC of the dissolution curve, it was evident that at the fixed SAC % cases, the interparticle cohesion reduction was more effective compared to those from the fixed silica weight %. The experimental evidence alludes that for fixed silica weight % cases, excess silica particles were available even after they completely covered the surface of the host particle (Ibu10), hence under a high energy mixing process (dry coating process/mechanical coating process), excess silica particles formed agglomerates with one another (Deng, Zheng, and Davé 2018); (Zheng et al. 2020).

The current study by the present inventors was able to demonstrate the benefit of employing silica mixtures that exhibit varying hydrophilicity, as the dry coating materials to induce both agglomerate reduction and expedited dissolution process. Based on the experimental results, the current study identified an additional factor to consider upon formulating a powder blend, such as the agglomeration of silica on the surface of the host particles. The present work provided tangible proof for the computational work by Deng et al. 2018 and Zheng et al. 2020 by clearly demonstrating better bulk properties improvement when the surface of API particles was not completely covered by silica (fixed SAC % cases) than the particles that were completely covered (fixed silica weight % cases).

Dry coating of APIs and/or excipients as shown herein offers an alternative to wet granulation and other methods to address processability of drug loaded formulations having cohesive and/or very cohesive particle sizes with potential for promoting direct compression tableting. The following examples are given to further illustrate the present invention. Again the invention is not limited to these examples. The examples are merely given to show the implementation of the present invention.

### EXAMPLES

### Example 1

### (Milled and Cohesive Poorly Water-Soluble API, Silica Amount Normalized by Weight Percent)

Synergistic effect from the hydrophobic and hydrophilic silica mixture when dry coating a fine and cohesive poorly water-soluble API-Agglomerate size reduction

Example 1 discusses reducing the agglomerate size of milled and cohesive poorly water-soluble BCS II classified drug after the dry coating with either single or binary components of nano-sized fumed silica.

Prior to the dry coating, an as-received coarse BCS II classified drug of mean particle size of >50 microns (hereon ibuprofen or Ibu, gift from BASF, USA) was milled down to a mean particle size of 10 microns using a fluidized energy mill (FEM, Pharmaceutical Micronizer Fluidized Energy Grinding Jet mill, Sturtevant Inc., Hanover, Massachusetts). The final milled size could be anything between 1 to 35 microns. For FEM milling, feeding rate, feeding pressure, and grinding pressure were set as 8 g/min, 30 psi, and 25 psi, respectively. Micronization could be done using other milling methods, including but not limited to conical milling or ball milling.

The milled Ibu was dry coated with the formulation listed in FIG. 15. Two different types of nano-sized fumed silica, Aerosil R972P (hydrophobic) and Aerosil A200 (hydrophilic) were used as the coating material, in which a single or mixture of the two was used. The amount of silica used for the dry coating was fixed based on the theoretically available milled Ibu surface area coverage (SAC) calculation, the value that ensured the Ibu surface's complete coverage [1, 2]. In FIG. 15, the associated % SAC and the weight percent (wt %) of silica used for the dry coating are listed as well. The solventless dry coating used a high-intensity vibratory mixer (LabRAM, Resodyn, USA). Based on the previously reported studies, the intensity of 75 times the gravitational force and 60 Hz for 5 minutes were selected for the operating parameters [3, 4]. The dry coating could be done with other methods investigated elsewhere [5-8]. A 300 mL screw top plastic container was used for the dry coating, mounted in LabRAM. For each dry coating run, about 66% of the container volume was taken up by powder, which comprised the milled Ibu and nano fumed silica. For the dry coating formulation, four cases were prepared. The uncoated and dry coated milled Ibu surfaces are shown in FIGS. 3(a) through 3(f), which present the Scanning Electron Microscopy (SEM, EM JSM-7900F, JEOL) analysis.

The primary particle size of API before and after dry coating and the resulted blends were evaluated using a compressed air dispersion method basis a laser diffraction particle sizer, Rodos/Helos (Sympatec Inc., NJ).

The agglomerate particle size distributions of the API before and after the dry coating and blends were evaluated using a gravity dispersion-based dynamic imaging particle sizer, Gradis/QicPic (Sympatec Inc., NJ). Unlike traditional sieving or SEM-imaging or optical microscope based agglomerate size estimation, employed Gradis/QiPic provides statistically representative and repeatable full agglomerate particle size distribution based on more than 107 particle image analysis while gentling dispersing the powders minimizing the impact on the agglomerates and preserving their original state as close as possible.

The summarized characteristic primary and agglomerated particle size distributions for the uncoated and dry coated APIs are shown in FIG. 16 Based on the evaluated primary and agglomerate particle size assessments, the agglomerate ratio (d_{50 Gradis}/d_{50 Rodos}) was found and listed in FIG. 16. As summarized in FIG. 16, notable agglomerate size reduction was observed for all dry coating cases. The milled Ibu, which was dry coated with binary silica mixture formulation, contains more A200 or single A200 formulation showed higher agglomerate size reduction due to greater surface area coverage, as shown in FIG. 15.

### Example 2

### (Milled and Cohesive Poorly Water-Soluble API, Silica Amount Normalized by Weight Percent):

Synergistic effect from the hydrophobic and hydrophilic silica mixture when dry coating a fine and cohesive poorly water-soluble API-Flowability improvements

Example 2 discusses the notable improvement in flowability for the cohesive and fine milled BCS II classified drug (Ibu) once the APIs are dry coated. Identical powder preparation and dry coating methods were used for Example 2, as discussed in Example 1.

Processability (flowability and bulk density) of the uncoated and dry coated API and the blends including the placebo were evaluated using a powder rheometer, FT4 (Freeman Technology, UK) under 3.0 kPa of pre-compaction pressure for shear testing. The containers used for bulk density and flowability measurements were acrylic cylinders (internal diameter 25 mm), 25 mL and 10 mL in volumes, respectively. The processability of the powders can also be assessed using a ring shear tester or other types of powder rheometer. The remarkable and flowability bulk density improvements after the dry coating of API were observed as shown in FIG. 17. The results were substantially different from uncoated API and using one flow agent alone as compared to using a binary flow agent as described herein, The flowability dynamics was listed in FIG. 17 as well as per Schulze scale describes [9, 10]. Once dry coated, the bulk density of the milled API was doubled, and the flowability of the milled API improved significantly, enhancing the flow dynamics from very cohesive to free flowing at maximum. In general, as the amount of silica was increased, the hydrophobic silica (R972P) led to greater flowability improvement than the cases with higher hydrophilic silica (A200) content. Nonetheless, in all cases, both the bulk powder density and flowability improved to support the continuous direct compaction. process However, combination of hydrophobic and hydrophilic silicas yielded the best outcomes.

### Example 3

### (Milled and Cohesive Poorly Water-Soluble API, Silica Amount Normalized by Weight Percent):

Synergistic effect from the hydrophobic and hydrophilic silica mixture when dry coating a fine and cohesive poorly water-soluble API-API dissolution rate improvement

Example 3 discusses the surprising API dissolution rate improvement after the dry coating with silica mixture which comprised of hydrophobic and hydrophilic silica. Identical powder preparation and coating methods were used for Example 3 as the previous examples.

To ensure that the powders are all wetted simultaneously without floating-issue, USP IV or flow-through-cell method was used for the API dissolution study (USP IV; Sotax, Switzerland). De-ionized water was used as the dissolution medium to maximize the discernment between the dry coating formulation. The temperature of the system and the dissolution medium flow rate through the 22.6 mm internal diameter of cells were fixed at 37° C. +0.2° C. and 16 mL/min during dissolution. Automated temporal sampling was done and analyzed using a Thermo Evolution UV spectrophotometer, which detects dissolved API concentration in real-time. Dissolved Ibu was detected at a selected wavelength of 221-nm, then quantified based on a pre-determined calibration curve. The measured solubility of Ibu in de-ionized water was 21 mg/L under ambient condition. Hence, by adding 15 mg of Ibu in each cell and dissolving them in 1 L of de-ionized water, the system held in non-sink condition.

Dissolution phenomenon is a convoluted process which is governed by the particle surface wetting rate and liquid penetration rate affected by particle size and surface hydrophobicity of the powder [12-15]. Therefore, along with the dissolution rate assessment, surface wettability of the uncoated and dry coated milled API was tested employing a liquid penetration testing or modified Washburn method [16].

Again, Washburn presents an equation which allows to evaluate the powder surface wettability as shown in Eq. (1):

$${m}^{2} = ( \frac{C {ρ}^{2} γcosθ}{η} ) t \qquad ( 2 )$$

In Eq. (2), η, p, and γ are the viscosity, the density, and the surface tension of the wetting liquid, respectively; C is a geometric packing factor; θ is the surface contact angle; m is the total mass of the wetting liquid that penetrates through the packed bed. The data is recorded in m² vs. t, forming a straight line with a slope ((C p2 γ cosθ)/η) [17]. As the testing liquid penetrates a packed bed of testing drug, the wettability of the drug (Thakker et al. 2013; Washburn 1921) was assessed.

Attention Sigma 700 (Biolin Scientifin, Linthicum) was employed to measure the liquid penetration rate through the drug powder-packed bed. The testing powder holder consists of a perforated cylindrical metallic tube (Height: 10 cm and ID: 2 cm) and a hook at the top of the cover equipped with screw threads. After placing a paper filter (pore size 20 to 25 pm) at the perforated end of the metallic tube, packed 0.8 g of testing powder before each measurement by a spring to ensure uniform packing between the samples. Positioned a 30 mL beaker containing a pre-saturated liquid (either n-hexane as a reference liquid or deionized water) below the perforated end of the tube. As the tip of the perforated end of the sample holder submerged (depth of submersion: 1.95 mm) in the liquid, Attention Sigma 700 (Biolin Scientific, Linthicum, MD, USA) recorded the mass of liquid penetrated the drug powder bed as a function of time.

Geometric packing factor, C, was determined for each powder formulation by employing the reference liquid, the drug saturated n-hexane [18, 19]. The reference liquid completely wets the particle surface, setting the surface contact angle to 0° (cos 0=1). The slope of the liquid penetration curve was used to calculate C [20, 21]. The same experimental steps were repeated with the dissolution medium (deionized water) as the testing liquid. Using the C value obtained from the test with the reference liquid and the slope of Eq. (1) from the test with the deionized water, the aqueous wettability, cosθ, was determined.

FIG. 19 presents the summarized powder surface wettability testing results. Unexpectedly, the surface wettability was highest when a 1 to 3 weight percent ratio of R972P (hydrophobic) to A200 (hydrophilic) silica mixture was used as the dry coating agent rather than the dry coating with A200 silica only. Such surprising improvement in the surface wettability is linked to the notable dissolution rate improvement, as shown in FIG. 2.

FIG. 2 presents the API dissolution rate evaluated for the uncoated and dry coated milled Ibu for 900 minutes of dissolution time. The study's most outstanding and unexpected outcome was the significant difference in dissolution rate improvement with the silica mixture containing the binary flow agents 0.58 wt % of R972P and 1.73 wt % of A200 (1 to 3 ratio). This result was substantially different than uncoated and coating with just one flow agent. The improvement was greatest even when compared to the A200-only coating. Apart from this notable finding, the combination of the agglomerate size reduction results shown in FIG. 16, the surface wettability results in FIG. 19 and dissolution rate results in FIG. 2 lead to the conclusion that the dissolution phenomenon is an interplay of the surface hydrophobicity and agglomerate size reduction. However, the 1 to 3 weight percent ratio of the hydrophobic and hydrophilic silica mixture dry coating on the milled API yielded the dissolution rate surpassing the hydrophilic silica-only dry coated API. This unanticipated result is the synergistic effect of more significant agglomerate size reduction from the hydrophobic silica and much-improved surface wettability from the hydrophilic silica.

### Example 4

### (Milled and Cohesive Poorly Water-Soluble API, Silica Amount Normalized by Available API's Surface Area Coverage (SAC) Percent):

Synergistic effect from the hydrophobic and hydrophilic silica mixture when dry coating a fine and cohesive poorly water-soluble API-Agglomerate size reduction

Example 4 discusses reducing the agglomerate size of milled and cohesive poorly water-soluble BCS II classified drug (ibuprofen, Ibu) after the dry coating with either single or binary components of nano-sized fumed silica. Based on the study presented by Kim et al. 2021, for the targeted normalized surface area coverage, a theoretical 50% SAC was selected, which resulted in most impactful dissolution rate improvement compared to the uncoated milled API.

Example 4 follows the identical sample preparation and testing methods described for Example 1. FIG. 20 presents the dry coating formulation details for Example 4. FIGS. 3(a) through 3(f) present the surface morphology of the milled Ibu before and after the dry coating from the Scanning Electron Microscopy (SEM, EM JSM-7900F, JEOL) analysis. After verifying that the dry coating was done successfully, the agglomerate particle size distribution of the prepared samples was evaluated and summarized in FIG. 21. For the primary particle size measurements, a compressed air dispersion-basis laser diffraction particle sizer, Rodos/Helos (Sympatec Inc., NJ) was used, with the dispersion pressure of 1.0 bar. For the agglomerate particle size measurement, a gravity dispersion-based dynamic imaging particle sizer, Gradis/QicPic (Sympatec Inc., NJ) was used. As summarized in FIG. 21, notable agglomerate size reduction was observed for all dry coating cases. The milled Ibu, which was dry coated with binary silica mixture formulation, contains more R972P or single R972P formulation showed higher agglomerate size reduction due to its less dispersive energy compared to A200.

### Example 5

### (Milled and Cohesive Poorly Water-Soluble API, Silica Amount Normalized by Available API's Surface Area Coverage (SAC) Percent):

Synergistic effect from the hydrophobic and hydrophilic silica mixture when dry coating a fine and cohesive poorly water-soluble API-Flowability improvements

Example 5 discusses the notable improvement in flowability for the cohesive and fine milled BCS II classified drug (Ibu) once the APIs are dry coated. Identical powder preparation and dry coating methods were used for Example 5, as discussed in Example 4.

Processability (flowability and bulk density) of the uncoated and dry coated API and the blends including the placebo were evaluated using a powder rheometer, FT4 (Freeman Technology, UK) under 3.0 kPa of pre-compaction pressure for shear testing. The containers used for bulk density and flowability measurements were acrylic cylinders (internal diameter 25 mm), 25 mL and 10 mL in volumes, respectively. The processability of the powders can also be assessed using a ring shear tester or other types of powder rheometer. The remarkable flowability and bulk density improvements after the dry coating of API were observed as shown in FIG. 22. Once dry coated, the bulk density of the milled API was doubled, and the flowability of the milled API improved significantly, enhancing the flow dynamics from very cohesive to free flowing at maximum. In general, as the amount of hydrophobic silica (R972P) used in the dry coating formulation had greater flowability improvement than the one with higher hydrophilic silica (A200) content. But in all case, both the bulk powder density and flowability improved to support the continuous direct compaction process [11].

### Example 6

### (Milled and Cohesive Poorly Water-Soluble API, Silica Amount Normalized by Available API's Surface Area Coverage (SAC) Percent):

Synergistic effect from the hydrophobic and hydrophilic silica mixture when dry coating a fine and cohesive poorly water-soluble API-API dissolution rate improvement

Example 6 discusses the surprising API dissolution rate improvement after the dry coating with silica mixture which comprised of hydrophobic and hydrophilic silica. Identical powder preparation and coating methods were used for Example 6 as the previous examples.

As described in Example 3, a USP IV (Sotax, Switzerland), flow-through-cell method was used as the dissolution testing device, where the dissolution medium of de-ionized water was flowing through at the rate of 16 mL/min. The temperature of the system was kept at 37° C. +0.2° C. The surface wettability of the prepared samples was evaluated by measuring the liquid penetration rate then calculating the surface contact angle as described in Example 3. For the surface wettability test, Attension sigma 700 (Biolin Scientific, Linthicum, MD, USA) was used.

FIG. 23 is the summarized surface wettability evaluation results. Unexpectedly, the surface wettability was highest when the milled Ibu was dry coated with the mixture of hydrophobic (R972P) and hydrophilic (A200) silica in the ratio of a 1 to 4 SAC % ratio. The second most wettable dry coated API was the one dry coated with purely A200 (hydrophilic) silica. The surprising phenomenon continued with the dissolution behavior, which is shown in FIG. 4.

FIG. 4 presents the API dissolution rate evaluated for the uncoated and dry coated milled Ibu for 900 minutes of dissolution time. The study's most outstanding and unexpected outcome was the significant difference in dissolution rate improvement with the silica mixture containing 10% SAC of R972P and 40% SAC of A200 (1 to 4 ratio). The substantially different improvement was greatest even when compared to the A200-only coating. Apart from this notable finding, the combination of the agglomerate size reduction results shown in FIG. 21, the surface wettability results in FIG. 23 and dissolution rate results in FIG. 4 lead to the conclusion that the dissolution phenomenon is an interplay of the surface hydrophobicity and agglomerate size reduction. However, the 1 to 3 weight percent ratio of the hydrophobic and hydrophilic silica mixture dry coating on the milled API yielded the dissolution rate surpassing the hydrophilic silica-only dry coated API. This unanticipated result is the synergistic effect of more significant agglomerate size reduction from the hydrophobic silica and much-improved surface wettability from the hydrophilic silica.

### Example 7

### (Milled and Cohesive Readily Water-Soluble API, Silica Amount Normalized by Available API's Surface Area Coverage (SAC) Percent):

Synergistic effect from the hydrophobic and hydrophilic silica mixture when dry coating a fine and cohesive readily water-soluble API-Agglomerate size reduction

Example 7 discusses reducing the agglomerate size of fine and cohesive readily water-soluble BCS I classified drug (micronized acetaminophen, mAPAP, donated from Mallinckrodt Inc., USA) after the dry coating with either single or binary components of nano-sized fumed silica. FIG. 24 presents the dry coating formulation. The dry coating was done following the description stated for Example 1. FIGS. 5(a) through 5(g) present the surface morphology of mAPAP and after the dry coating from the Scanning Electron Microscopy (SEM, EM JSM-7900F, JEOL) analysis.

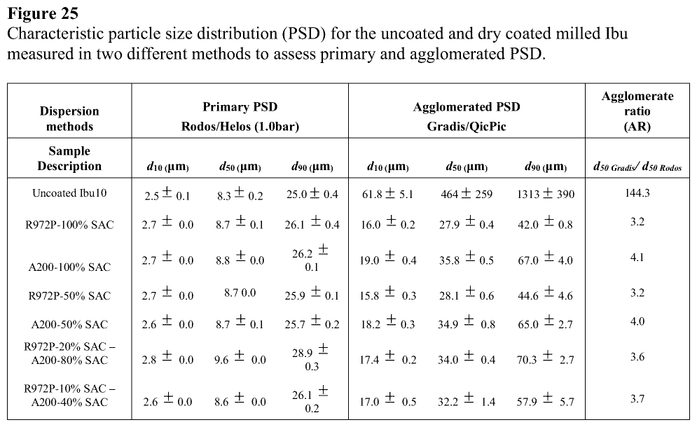

Verifying that the dry coating was done successfully, the agglomerate particle size distribution of the prepared samples was evaluated and summarized in FIG. 25. For the primary particle size measurements, a compressed air dispersion-basis laser diffraction particle sizer, Rodos/Helos (Sympatec Inc., NJ) was used, with the dispersion pressure of 1.0 bar. For the agglomerate particle size measurement, a gravity dispersion-based dynamic imaging particle sizer, Gradis/QicPic (Sympatec Inc., NJ) was used. As summarized in FIG. 25, notable agglomerate size reduction was observed for all dry coating cases. The milled Ibu, which was dry coated with binary silica mixture formulation, contains more R972P or single R972P formulation showed higher agglomerate size reduction due to its less dispersive energy compared to A200.

### Example 8

### (Milled and Cohesive Readily Water-Soluble API, Silica Amount Normalized by Available API's Surface Area Coverage (SAC) Percent):

Synergistic effect from the hydrophobic and hydrophilic silica mixture when dry coating a fine and cohesive poorly water-soluble API-Flowability improvements

Example 8 discusses the notable improvement in flowability for the cohesive and fine BCS I classified drug (mAPAP) once the APIs are dry coated. Identical powder preparation and dry coating methods were used for Example 8, as discussed in Example 7.

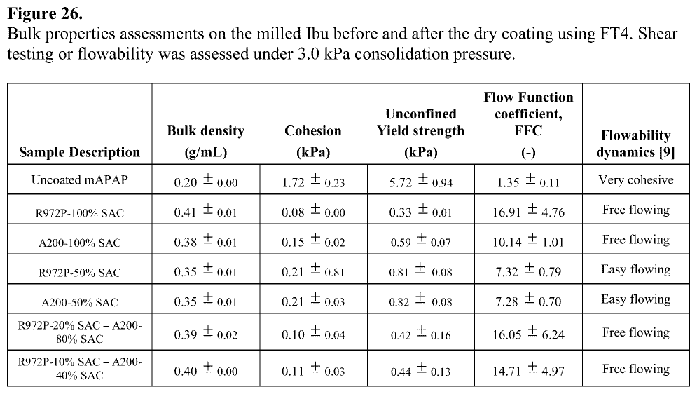

Processability (flowability and bulk density) of the uncoated and dry coated API and the blends including the placebo were evaluated using a powder rheometer, FT4 (Freeman Technology, UK) under 3.0 kPa of pre-compaction pressure for shear testing. The containers used for bulk density and flowability measurements were acrylic cylinders (internal diameter 25 mm), 25 mL and 10 mL in volumes, respectively. The processability of the powders can also be assessed using a ring shear tester or other types of powder rheometer. The remarkable flowability and bulk density improvements after the dry coating of API were observed as shown in FIG. 26. Once dry coated, the bulk density of the milled API was doubled, and the flowability of the milled API improved significantly, enhancing the flow dynamics from very cohesive to free flowing at maximum. In general, as the amount of hydrophobic silica (R972P) used in the dry coating formulation had greater flowability improvement than the one with higher hydrophilic silica (A200) content. But in all case, both the bulk powder density and flowability improved to support the continuous direct compaction process [11].

### Example 9

### (Milled and Cohesive Readily Water-Soluble API, Silica Amount Normalized by Available API's Surface Area Coverage (SAC) Percent):

Synergistic effect from the hydrophobic and hydrophilic silica mixture when dry coating a fine and cohesive readily water-soluble API-API release rate improvement from a 40% API loaded 12.7 mm tablet of 200 mg compressed to 155 MPa in a pH 5.8 PBS buffer

Example 9 discusses the API release rate improvement due to the synergistic impact of the hydrophobic and hydrophilic silica mixture, mainly when the silica mixture completely covers the available surface area of the API. To prepare the testing samples, a blend comprised of uncoated or dry coated API, as listed in FIG. 24, was blended with Avicel PH105 and Pharmatose 450. The API loading of the blend was fixed to 40 wt %, while Avicel PH105 and Pharmatose 450 took 30 wt % each. A total of 90 g of the blend was mixed in a 600 mL V-shaped acrylic container for 16 minutes to ensure well mixing. The rotational speed was fixed to 25 rpm. Then, using a sampling riffler, powders were sampled prior to generate 200 mg tablets using a manual Carver platen press (Carver, Inc., USA). The employed die's internal diameter was 12.7 mm in size.

Prepared tablets were then evaluated for their API release rates by using a USP II (Sotax, Switzerland) paddle method dissolution apparatus. As the dissolution medium, a pH 5.8 PBS buffer was used. The rotation speed of the paddle was kept at 50 rpm while the dissolution vessel's temperature was held at 37° C. to 0.2° C. The volume of the dissolution medium was 600 mL. At a pre-determined time-points, 3 mL of sample was drawn and filtered, which was diluted by adding 10 mL of the buffer solution. When 3 mL of sample was drawn, 3 mL of makeup medium was added as well.

FIGS. 6 (a) and 6 (b) are the summarized API release profile from the tested 200 mg tablets. For each formulation, a minimum of six to maximum of twelve tablets were evaluated to ensure repeatability of the API release rate. In both cases, the API release rate was fastest with the tablets containing the binary silica coated API, especially at the early phase of the dissolution process. This is a notable and unexpected find, which implies that even with the readily soluble API, the synergic effect from the reduced agglomerate size and improved surface wettability can further expediate its dissolution rate. Hence, such strategy can potentially expand the formulation of the immediate release pharmaceutical products, without having change the API delivery platform from solid to liquid or suspension.

### Example 10

Example 10 assesses the bulk density, FFC, and tensile strength of uncoated and dry coated microcrystalline cellulose-based excipients.

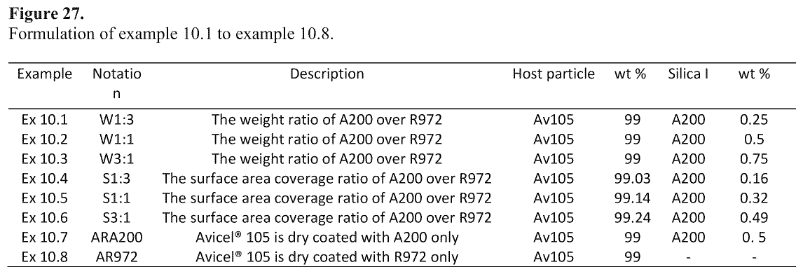

As received excipient, Avicel® 105, was dry coated with specific amount of Aerosil A200 (A200) and/or Aerosil R972P (R972) using a high-intensity vibratory mixer (LabRAM, Resodyn, USA) at the intensity of 70 times the gravitational force and 60 Hz for 5 minutes. The dry coating could be done with other methods which were investigated in elsewhere [5-8]. A 300 mL screw top plastic container was used for the dry coating. For each dry coating run, about 66% of the container volume was taken up by powder, which comprised the A200, R972, and excipients. For the dry coating formulation used in Example 10 are shown in FIG. 27.

Processability (flowability and bulk density) of the dry coated excipients were evaluated using a powder rheometer, FT4 (Freeman Technology, UK) under 3.0 kPa of pre-compaction pressure for shear testing. The containers used for bulk density and flowability measurements were acrylic cylinders (internal diameter 25 mm), 25 mL and 10 mL in volumes, respectively. The processability of the powders can also be assessed using a ring shear tester or other types of powder rheometer. The suitability of an excipient intended for a tablet dosage form can be evaluated by its tabletability, compressibility, and compactability. Tabletability, i.e., tablet tensile strength as a function of compaction pressure, is the capability of a powder to gain strength under pressure, and useful to evaluate manufacturability. The tablet tensile strength was evaluated with a texture analyzer (Texture Technologies Corp., USA). Carver platen press (Carver, Inc., USA) with a 0.5-inch inner diameter stainless die and a flat-faced round punch was used to prepare 500 mg tablets under 1.5 metric ton (equivalent to 114 MPa) compaction pressure.

SEM micrographs (FIGS. 7A-7D, not all cases are shown here) indicated coating of guest material is evident. Even when the excipient has irregular shape, dry coating of guest material is good and visible on the surface without appreciable agglomeration of the guest material.

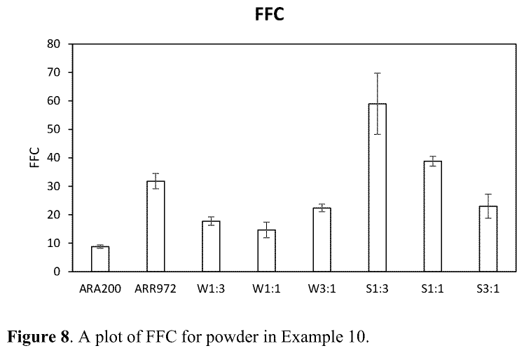

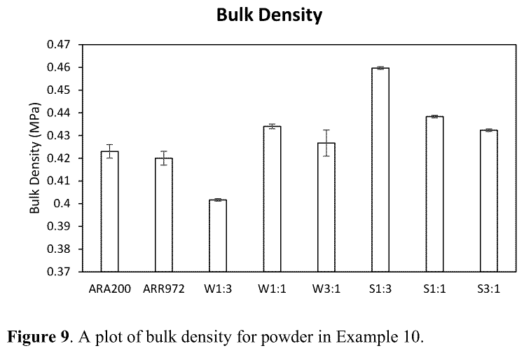

FIG. 8 depicts FFC for the as received and dry coated excipient blends listed in FIG. 27. The bars in FIG. 8, FIG. 9, and FIG. 10 are labeled as W1:3 (Ex 13.1, Avicel® 105 was dry coated with A200 and R972 at weight ratio 1:3), W1:1 (Ex 13.2, Avicel® 105 was dry coated with A200 and R972 at weight ratio 1:1), W3:1 (Ex 13.3, Avicel® 105 was dry coated with A200 and R972 at weight ratio 3:1), S1:3 (Ex 13.4, Avicel® 105 was dry coated with A200 and R972 at surface area coverage ratio 1:3), S1:1 (Ex 13.5, Avicel® 105 was dry coated with A200 and R972 at surface area coverage ratio 1:1), S3:1 (Ex 13.6, Avicel® 105 was dry coated with A200 and R972 at surface area coverage ratio 3:1), ARA200 (Ex 13.7, Avicel® 105 was dry coated with 1 wt % A200), and ARR972 (Ex 13.8, Avicel® 105 was dry coated with 1 wt % R972). As shown in FIG. 8, Avicel® 105 dry coated with 1 wt % of A200 is close to free flowing (FFC>10 is characterized as free flowing), while R972 can lead to better flowability. Compared with ARR972, the loss of FFC happens when binary silica coating was included in W1:3, W1:1, W3:1, and S3:1. What out of surprise is that S1:3 and S1:1 have higher FFC than ARR972, which indicates the binary silica coating can bring higher performance than single silica coating.

FIG. 9 depicts bulk density for the as received and dry coated excipient blends listed in FIG. 27. As shown in FIG. 9, except W1:3, most of binary silica coated excipients gain bulk density improvement over single silica coating. Bulk density enhancements show largest gain for S1:3, followed by S1:1, W1:1, S3:1, and W3:1. It should be noted that S1:3 and S1:1 achieve improved FFC and bulk density compared with single silica coating.

FIG. 10 depicted tensile strength under 1.5 metric ton compression force of dry coated excipients with formulation shown in FIG. 27. As shown in FIG. 10, dry coated Avicel® 105 with 1 wt % A200 (ARA200) has highest tensile strength and with 1 wt % R972 (ARR972) has lowest tensile strength. Binary silica coating shows various loss of tensile strength compared to ARA200, while S1:1 has almost similar tensile strength compared to. ARA200. Considering FFC and bulk density, it is surprising that S1:1 can not only achieve great improvement in bulk density and FFC, but also keep high tensile strength.

Example 10 demonstrates that binary silica coating can lead to unpredictable improvement in bulk density, FFC while keep high tensile strength over as received excipients in terms of flowability and bulk density.

### Example 11

Example 11 assesses the bulk density, FFC and tensile strength of several blends using milled Pharmacel® 101 and milled and coated Pharmacel® 101.

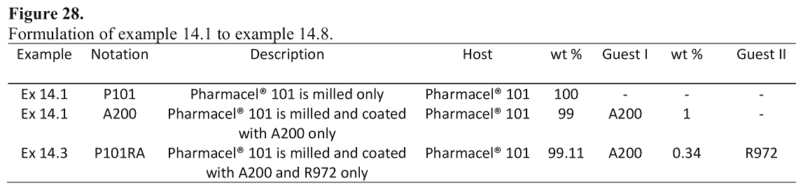

The coating formulation used in Example 11 are shown in FIG. 28. Prior to the dry coating, the powder blend was mixed via a 4-pint V-shaped container for 10 min at 25 rpm. The blending could be done with any other method. Upon preparing blends, mixing parameters such as the order of filling each constituent, mixing intensity, and mixing time were held constant.

As received excipient, Pharmacel® 101, was milled only or simultaneously milled and coated with specific amount of Aerosil A200 (A200) and/or Aerosil R972P (R972) using a fluidized energy mill (FEM, Pharmaceutical Micronizer Fluidized Energy Grinding Jet mill, Sturtevant Inc., Hanover, Massachusetts). The final milled size could be around 35 microns. For FEM milling, feeding rate, feeding pressure, and grinding pressure were set as 2 g/min, 30 psi, and 25 psi, respectively.

After milling process via FEM, the coated or uncoated excipients are blended with 60% micronized Acetaminophen (CAPAP). The powder blend was mixed via a 4-pint V-shaped container for 12 min at 25 rpm. The blending could be done with any other methos. Upon preparing blends, mixing parameters such as the order of filling each constituent, mixing intensity, and mixing time were held constant.

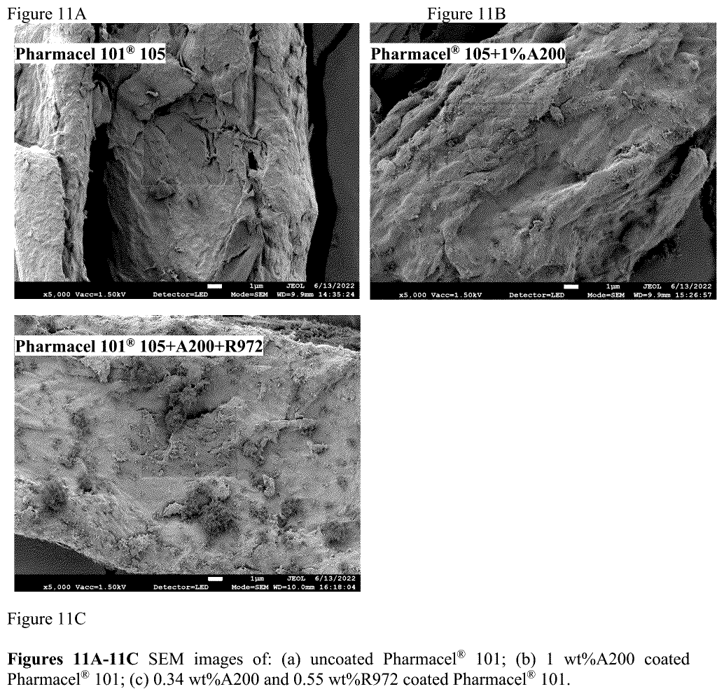

SEM micrographs (FIGS. 11A-11C) indicated binary silica coating and single silica coating have similar silica distribution on the surface of Pharmacel® 101.

The primary particle size of excipients before and after dry were evaluated using a compressed air dispersion method basis a laser diffraction particle sizer, Rodos/Helos (Sympatec Inc., NJ). The particle size (d₅₀) is shown in FIG. 28

Processability (flowability and bulk density) of the dry coated excipients were evaluated using a powder rheometer, FT4 (Freeman Technology, UK) under 3.0 kPa of pre-compaction pressure for shear testing. The containers used for bulk density and flowability measurements were acrylic cylinders (internal diameter 25 mm), 25 mL and 10 mL in volumes, respectively. The processability of the powders can also be assessed using a ring shear tester or other types of powder rheometer. The suitability of an excipient intended for a tablet dosage form can be evaluated by its tabletability, compressibility, and compactability. Tabletability, i.e., tablet tensile strength as a function of compaction pressure, is the capability of a powder to gain strength under pressure, and useful to evaluate manufacturability. The tablet tensile strength was evaluated with a texture analyzer (Texture Technologies Corp., USA). Carver platen press (Carver, Inc., USA) with a 0.5-inch inner diameter stainless die and a flat-faced round punch was used to prepare 500 mg tablets under 1.5 metric ton (equivalent to 114 MPa) compaction pressure.

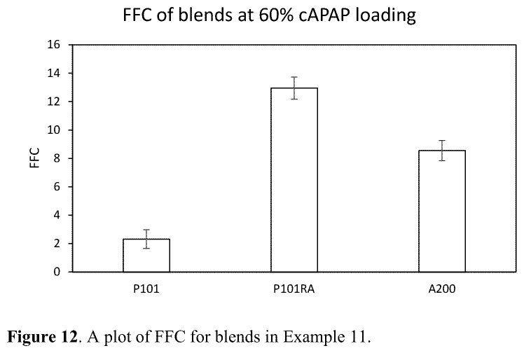

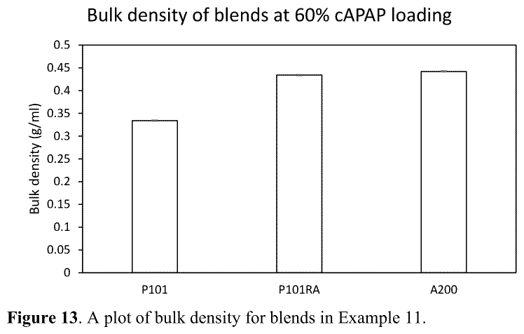

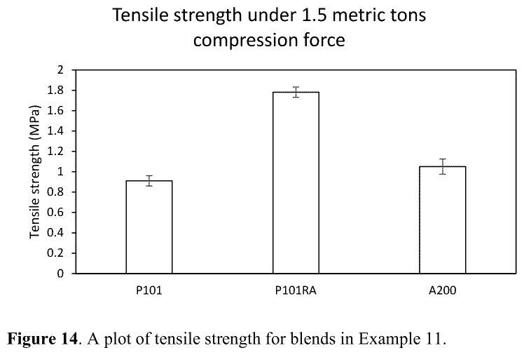

FIG. 12 depicts FFC of the 60% CAPAP loading blends with excipients listed in FIG. 28. The bars in FIG. 12, FIG. 13 and FIG. 14 are labeled as P101, P101RA, A200, which mean the 60% cAPAP loading blends containing milled and uncoated Pharmacel® 101, milled and coated Pharmacel® 101 with 0.34 wt % A200 and 0.55 wt % R972, and milled and coated Pharmacel® 101 with 1 wt % A200, respectively. FIG. 13 depicts the improved bulk density after dry coating. Binary silica coated blend has highest tensile strength, and the blends is characterized as free flowing.

FIG. 13 depicts bulk density of the 60% cAPAP loading blends with excipients listed in FIG. 28. FIG. 13 depict the improved bulk density after dry coating. Binary silica coated and single silica coated blend have similar bulk density

FIG. 14 depicts tensile strength under 1.5 metric ton compression force of the 60% cAPAP loading blends with excipients listed in FIG. 28. As shown in FIG. 14, after dry coating, the blend with A200 has slight improvement in tensile strength. However, the binary silica coated blend shows a great improvement in tensile strength after dry coating.

Overall, it is surprising that binary silica coating shows higher improvement in tensile strength and FFC compared to single silica coating, while there is no compromise on enhancement of bulk density.

Again, the results shown herein demonstrate that a dry coated the dry coating of API and/or excipients for improved blend properties such as tablet dissolution due to the agglomerate size reduction along with improved blend flowability and bulk density without adverse impact on tablet compaction, implying enhanced blend processability.

The impact of dry coating with two diverse types and amounts of silica was systematically assessed on cohesion, agglomeration, flowability, bulk density, wettability, and surface energy of fine milled ibuprofen (˜10 μm). Agglomerated sizes were measured via gentler gravity-based dispersion, resulting in excellent size resolution.

All dry coated ibuprofen powders exhibited dramatic agglomeration reduction, decreased cohesion, unconfined yield strength, and improved flowability, which are attributed to the nano-scale surface morphology imparted by the binary silica coating. Their blends exhibited profound enhancement in flowability and bulk density even at low API loadings, as well as the content uniformity for the lowest drug loading. Moreover, hydrophobic silica coating improved drug dissolution rate without appreciably reducing tablet tensile strength.

Definition of averaged or predicted FFC based on the component FFC values:

The weighted average FFC in a blend using the fractional surface area as weights is described here. Since FFC is a non-linear measure, the procedure outlined below could be conveniently modified but it is generally adequate after capping any FFC>10 to be equal to 10. For simplicity, the particle diameter for species i is taken to be its D50 size. The method comprises estimating the weight of a single particle for each component in the blend based on their densities and particle sizes, calculating the total number of particles of each component based on the mixed quantities, and determining the total surface area of particles for each component. A simplification based on algebraic manipulation can be also done.

The fractional surface area of each component is then determined as the ratio of each component's total surface area to the entire blend's total surface area. These fractional surface areas serve as weights in the weighted averaging of FFC for each component in the blend, thereby offering a more accurate and representative FFC of blends which is dominated by the presence of fine component, whose representation may be more accurate by their more surface area weight fractions compared to coarser components.

Overall, the weighted average FFC in a blend using fractional surface area as weights may be estimated as follows, where any component FFC>10 is capped and made equal to 10:

Step 1. Estimating the weight (Wᵢ) of a single particle for each component in the blend using the formula for the volume of a sphere and the density (pᵢ) of each component. For ‘i’ components, the weights would be calculated as follows: Wᵢ=4/3* pᵢ*(Dᵢ/2)∧3*pᵢ

Step 2. Calculating the total number of particles (Ni) of each component in the blend, based on the mass quantities in which the components are mixed, Qᵢ: Nᵢ=Qᵢ/Wᵢ; where ΣWᵢ=W, total weight

Step 3. Determining the total surface area (Aᵢ) of particles for each component using the formula for the surface area of a sphere: Aᵢ=Nᵢ*4*pᵢ*(Dᵢ/2)∧2

Step 4. One could simplify the above and directly obtain A1=(6*Qᵢ)/(Dᵢ*pᵢ) hence this would be the first step, followed by next two steps

Step 5. Calculate the fractional surface area (Fᵢ) of each component as the ratio of each component's total surface area to the blend's total surface area. For each component ‘i’, this would be calculated as: Fᵢ=Aᵢ/E (Aᵢ) for all ‘i’

Step 6. Finally, using the fractional surface areas as weights in the weighted averaging of FFC for each component in the blend: FFC_avg=>Σ(Fᵢ*FFCᵢ) for all ‘i’.

FFC values ranged from 2.2 to 2.4, which are cohesive but almost very cohesive. However, the actual FFC due to the claimed novelty of our invention, ranged much higher, from 5.4 to as high as 7.1.

While exemplary embodiments have been described herein, it is expressly noted that these embodiments should not be construed as limiting, but that additions and modifications to what is expressly described herein also are included within the scope of the invention. For example, the systems and processes are applicable to other excipients.

Moreover, it is to be understood that the features of the various embodiments described herein are not mutually exclusive and can exist in various combinations and permutations, even if such combinations or permutations are not made express herein, without departing from the spirit and scope of the invention.

### REFERENCES

ADDIN EN. REFLIST 1. Yang, J., et al., Dry particle coating for improving the flowability of cohesive powders. Powder Technology, 2005. 158 (1-3): p. 21-33.
2. Chen, Y., et al., Fluidization of coated group C powders. AIChE Journal, 2008. 54 (1): p. 104-121.
3. Huang, Z., et al., Improving blend content uniformity via dry particle coating of micronized drug powders. European Journal of Pharmaceutical Sciences, 2017. 104: p. 344-355.
4. Chen, L., et al., Surface engineered excipients: II. Simultaneous milling and dry coating for preparation of fine-grade microcrystalline cellulose with enhanced properties. International Journal of Pharmaceutics, 2018. 546 (1-2): p. 125-136.
5. Watano, S., et al., Fine particle coating by a novel rotating fluidized bed coater. Powder Technology, 2004. 141 (3): p. 172-176.
6. Han, X., et al., Simultaneous micronization and surface modification for improvement of flow and dissolution of drug particles. International Journal of Pharmaceutics, 2011. 415 (1-2): p. 185-195.
7. Jallo, L. J., et al., Improvement of flow and bulk density of pharmaceutical powders using surface modification. International Journal of Pharmaceutics, 2012. 423 (2): p. 213-225.
8. Chen, L., et al., Fine grade engineered microcrystalline cellulose excipients for direct compaction: Assessing suitability of different dry coating processes. European Journal of Pharmaceutical Sciences, 2020. 151.
9. Freeman, R., Measuring the flow properties of consolidated, conditioned and aerated powders-A comparative study using a powder rheometer and a rotational shear cell. Powder Technology, 2007. 174 (1-2): p. 25-33.
10. Schulze, D., J. Schwedes, and J. W. Carson, Powders and bulk solids: Behavior, characterization, storage and flow. Powders and Bulk Solids: Behavior, Characterization, Storage and Flow. 2008. 1-511.
11. Sun, C. C., Setting the bar for powder flow properties in successful high speed tableting. Powder Technology, 2010. 201 (1): p. 106-108.
12. Alway, B., R. Sangchantra, and P. J. Stewart, Modelling the dissolution of diazepam in lactose interactive mixtures. International Journal of Pharmaceutics, 1996. 130 (2): p. 213-224.
13. Jinno, J. I., et al., Effect of particle size reduction on dissolution and oral absorption of a poorly water-soluble drug, cilostazol, in beagle dogs. Journal of Controlled Release, 2006. 111 (1-2): p. 56-64.
14. Forny, L., A. Marabi, and S. Palzer, Wetting, disintegration and dissolution of agglomerated water soluble powders. Powder Technology, 2011. 206 (1-2): p. 72-78.
15. Kim, S., E. Bilgili, and R. N. Dave, Impact of altered hydrophobicity and reduced agglomeration on dissolution of micronized poorly water-soluble drug powders after dry coating. International Journal of Pharmaceutics, 2021. 606.
16. Washburn, E. W., The dynamics of capillary flow. Physical Review, 1921. 17 (3): p. 273-283.
17. Llusa, M., et al., Measuring the hydrophobicity of lubricated blends of pharmaceutical excipients. Powder Technology, 2010. 198 (1): p. 101-107.
18. Thakker, M., et al., Wettability measurement apparatus for porous material using the modified Washburn method. Measurement Science and Technology, 2013. 24 (12).
19. Li, M., et al., A comparative assessment of nanocomposites vs. amorphous solid dispersions prepared via nanoextrusion for drug dissolution enhancement. European Journal of Pharmaceutics and Biopharmaceutics, 2017. 119: p. 68-80.
20. Neirinck, B., et al., Wettability assessment of submicrometer 20. alumina powder using a modified Washburn method. Journal of the American Ceramic Society, 2010. 93 (9): p. 2515-2518.
21. Steele, D. F., et al., Surface energy of microcrystalline cellulose determined by capillary intrusion and inverse gas chromatography. AAPS Journal, 2008. 10 (3): p. 494-503.

1. A method of using binary coating agents in powder coating for cohesive or very cohesive particles, comprising:
taking one or more powder components having a flow function coefficient (FFC) under 4 FFC prior to any surface modification or treatment, wherein at least one of the components is an active pharmaceutical ingredient (API) or an excipient;
dry coating by a binary dry coating with both a hydrophobic dry coating agent and a hydrophilic dry coating agent on a surface of the API or the excipient or both the API and the excipient for forming a binary dry coated component,
using for the hydrophobic dry coating agent and the hydrophilic dry coating agent a weight percentage ratio or a ratio of the normalized surface area coverage (SAC) for the dry coated component, and
providing by the binary dry coating flowability, bulk density, deagglomeration, and dissolution properties that are substantially different for the binary dry coated component and/or a blend containing the binary dry coated component as compared to using the hydrophobic or the hydrophilic dry coating agents alone or no dry coating agents used.

2. The method of claim 1, wherein the hydrophobic dry coating agent is a hydrophobic nano-fumed silica and the hydrophilic dry coating agent is hydrophilic nano-fumed silica, and wherein the normalized surface area coverage (SAC) ratio of 1 to 4 with the dry coating is obtained.

3. The method of claim 1, wherein the dry coated component is used in a blend for providing for providing flowability, bulk density, deagglomeration, and dissolution of the blend that is increased as compared to using the hydrophobic or the hydrophilic dry coating agents alone or no dry coating agents used.

4. The method of claim 1, wherein a total weight percent of dry coating agents is less than 1 wt %.

5. The method of claim 1, further comprises combining the hydrophobic and the hydrophilic dry coating agents to dry coat an individual powder or a component within a multi-component blend at a weight percentage with respect to the coated component (wt %) of 0.01 wt % to 2.31.

6. The method of claim 5 wherein the hydrophobic and the hydrophilic dry coating agents are a hydrophobic nano-fumed silica microcrystalline cellulose (MCC) (Aerosil® R972P) and hydrophilic nano-fumed silica microcrystalline cellulose (MCC) (Aerosil® A200).

7. The method of claim 1, wherein the dry coating further includes using a functionalized hydrophobic and hydrophilic silicas as the dry coating agents in an amount at least 0.01 wt % in the blend.

8. The method of claim 7, wherein the dry coated component has a fine particle size defined in a range of 10 μm-15 μm.

9. The method of claim 1, wherein the dry coating agent is silica within a range of about 1 wt % to about 2.31 wt % with respect to the coated component, and the dry coating agent is both a hydrophobic fumed silica (Aerosil R972P) with a specific surface area of 90-130 m²/g, and a hydrophilic fumed silica with a specific surface area of 200 m²/g (Aerosil 200).

10. A method of using binary coating agents in powder coating for cohesive or very cohesive particles, comprising:
taking one or more fine powder cohesive or very cohesive components having a particle size of less than 50 microns and having a flow function coefficient (FFC) under 4 FFC prior to any surface modification or treatment, wherein at least one of the components is an active pharmaceutical ingredient (API) or excipient;
simultaneously dry coating with both a hydrophobic dry coating agent and a hydrophilic dry coating agents on a surface of the API or the excipient or both the API and the excipient for forming a dry coated component,
obtaining a normalized surface area coverage (SAC) ratio of 1 to 4 with the dry coating; and
providing by the binary dry coating flowability, bulk density, deagglomeration, and dissolution properties that are substantially different for the binary dry coated component and/or a blend containing the binary dry coated component as compared to using the hydrophobic or the hydrophilic dry coating agents alone or no dry coating agents used.

11. The method of claim 10, wherein the hydrophobic dry coating agent and the hydrophilic dry coating agent are a hydrophobic nano-fumed silica microcrystalline cellulose (MCC) Aerosil® (R972P) and a hydrophilic nano-fumed silica (Aerosil® A200) simultaneously dry coated onto a surface of the active pharmaceutical ingredient (API) or the excipient powders or both the active pharmaceutical ingredient (API) and the excipient powders.

12. The method of claim 10 wherein a ratio of the hydrophobic dry coating agent and the hydrophilic dry coating agent has a weight percent ratio of between 1 to 3 and 3 to 1.

13. The method of claim 10, where the API is a single API, and the dry coating agents are a hydrophobic silica and a hydrophilic silica.

14. The method of claim 13, wherein the dry coating provides a drug dissolution rate of a blend higher than that compared to an untreated blend.

15. The method of claim 10, wherein a ratio of the hydrophobic dry coating agent and the hydrophilic dry coating agent has a weight percent ratio of between 1 to 3 and 5 to 1.

16. The method of claim 15, wherein the flow aid is selected from a group consisting of magnesium stearate (MgSt), Leucine, stearic acid, Sodium Lauryl Sulfate (SLS), Sodium Dodecyl Sulfate (SDS), and any combination thereof.

17. The method of claim 10, wherein the hydrophobic dry coating agent and the hydrophilic dry coating agent are used in a blend for a pharmaceutical composition.

18. A method of using binary coating agents in powder coating for cohesive or very cohesive particles, comprising:
taking one or more fine powder cohesive or very cohesive components having a particle size of less than 50 microns and having a flow function coefficient (FFC) under 4 FFC prior to any surface modification or treatment, wherein at least one of the components is an active pharmaceutical ingredient (API) or an excipient;
simultaneously dry coating with both a hydrophobic silica and a hydrophilic dry silica on a surface of the API or the excipient or both the API and the excipient for forming a dry coated component,
using the hydrophobic silica and the hydrophilic silica agent at a weight percent ratio between 1 to 3 and 5 to 1, and obtaining a normalized surface area coverage ratio of 1 to 4 with the dry coating; and
providing by the binary dry coating flowability, bulk density, deagglomeration, and dissolution properties that are substantially different for the binary dry coated component and/or a blend containing the binary dry coated component as compared to using the hydrophobic or the hydrophilic dry coating agents alone or no dry coating agents used.

19. The method of claim 18, wherein the API is a milled and cohesive BCS I or BCS II classified drug and improved bulk density, powder flow, and agglomerate size reduction for the milled and cohesive BCS I or BCS II classified drug was obtained in a comparable or better range than a single component silica dry coating.

20. The method of claim 18, wherein the hydrophobic and hydrophilic silicas are hydrophobic nano-fumed silica (Aerosil® R972P) and hydrophilic nano-fumed silica (Aerosil® A200) that are both mixed together and simultaneously dry coated onto the surface of an API, excipient, or both an API and excipient.

21. The method of claim 1, wherein a ratio of the hydrophobic dry coating agent and the hydrophilic dry coating agent has a weight percent ratio of between 1 to 3 for dry coating the API and dissolution as well as blend flowability increase.

22. The method of claim 1, wherein a ratio of the hydrophobic dry coating agent and the hydrophilic dry coating agent has a weight percent ratio of between 5 to 1 for dry coating the excipient and tensile strength increase of a tablet made therefrom as well as blend flowability increase.

23. The method of claim 1, wherein the substantially different property for the binary dry coated component and/or the blend containing the binary dry coated component is an API dissolved weight percentage of at least 70 wt % after a dissolution time of 100 minutes.

24. The method of claim 1, wherein the substantially different property for the binary dry coated component and/or the blend containing the binary dry coated component is a flow function coefficient (FFC) greater than 10 FFC.
